(19) United States
(12) Patent Application Publication
(10) Pub. No.: US 2017/0166920 A1
(43) Pub. Date: Jun. 15, 2017

(54) PLANTS WITH ENHANCED RESISTANCE TO PHYTOPHTHORA

(71) Applicant: TWO BLADES FOUNDATION, Chicago, IL (US)

(72) Inventor: Sophien Kamoun, Norwich (GB)

(21) Appl. No.: 15/115,498

(22) PCT Filed: Jan. 28, 2015
(86) PCT No.: PCT/US2015/013287

§ 371 Date: Jul. 29, 2016

Related U.S. Application Data
(60) Provisional application No. 61/933,548, filed on Jan. 30, 2014.

(51) Int. Cl. C12N 15/82; C07K 14/415

(52) U.S. Cl. CPC C12N 15/8282; C07K 14/415

(57) ABSTRACT

Methods and compositions for enhancing the resistance of plants to oomycete plant pathogens are provided. The methods involve decreasing in the plant or part thereof the level of a remorin, particularly a remorin that is known to occur in the extrahaustorial membrane that is formed in a host plant in response to an infection by one or more oomycete plant pathogens. Compositions comprise plants and plant cells with a reduced level and/or activity of at least one remorin in the plant or part thereof when compared to a control plant or part thereof. Additionally provided are methods for using the plants in agriculture to limit diseases caused by oomycete pathogens

### CROSS-REFERENCE TO RELATED APPLICATION

This application claims the benefit of U.S. Provisional Application No. 61/933,548, filed Jan. 30, 2014, which is hereby incorporated herein in its entirety by reference.

### REFERENCE TO A SEQUENCE LISTING SUBMITTED AS A TEXT FILE

The official copy of the sequence listing is submitted electronically via EFS-Web as an ASCII formatted sequence listing with a file named 070294.0074SEQLST.TXT, created on Jan. 28, 2015, and having a size of 13.6 kilobytes, and is filed concurrently with the specification. The sequence listing contained in this ASCII formatted document is part of the specification and is herein incorporated by reference in its entirety.

### FIELD OF THE INVENTION

The present invention relates to the field of plant improvement, particularly to methods for making and using plants with enhanced resistance to oomycete pathogens.

### BACKGROUND OF THE INVENTION

Late blight is one of the most devastating diseases affecting potato (Solanum tuberosum) production worldwide. This disease is caused by the oomycete plant pathogen, Phytophthora infestans. Traditional approaches to combating this plant pathogen involve the application of chemical pesticides to potato plants and the use of late blight resistant potato varieties which possess a resistance (R) gene. Potato breeders have introduced at least 11 late blight resistance alleles of the R3a gene rom Solanum demissum into the cultivated potato (Gebhardt and Valkonen (2001) Annu. Rev. Phytopathol. 39:79-102). The products of R alleles recognize the products of corresponding alleles of AvrR3a in races of P. infestans, triggering disease resistance and a programmed cell-death in the vicinity of the pathogen attack, that has been termed the hypersensitive response (HR). For this type of resistance, recognition depends on the interaction between R gene product in the plant and a corresponding dominant Avr gene product expressed by the invading phytopathogen. The key difficulty with this type of resistance is that the host plant must possess an R allele of R3a that corresponds to the Avr3a allele of a particular P. infestans race that is infecting the host plant. Otherwise, the host plant will not display resistance to that race of P. infestans. Given that new races of P. infestans with new alleles of AvrR3a continue to appear, it is an ongoing challenge for potato breeders first to find new R alleles of R3a that correspond to the new alleles of AvrR3a and then to breed new potato varieties with these new R alleles. Because such approaches for producing potato plants with enhanced resistance to P. infestans are both time consuming and costly, new strategies for producing crop plants that are resistance to this devastating plant pathogen are desired.

New strategies for producing crop plants with enhanced resistance to P. infestans and other oomycete pathogens may result from gaining a better understanding of the host-pathogen interactions. The infection of a plant by a pathogen initiates a cascade of alterations in the host plant including, for example, local and systemic changes in gene and metabolic processes as well as structural changes in the host plant cells that are adjacent to or in the vicinity of invading pathogen. The most devastating plant pathogens, including the oomycetes Phytophthora and downy mildews, powdery mildew fungi, and rust fungi, form specialized infection structures called haustoria. Haustoria are intracellular projections of the pathogen hyphae into host cells enveloped by a perimicrobial membrane called the extrahaustorial membrane (EHM). Haustoria serve as feeding structures (Voegele et al., 2001, PNAS 98:8133-8138) and are thought to be critical for the delivery of effectors into host cells (Whisson et al. (2007) Nature 450:115-118; Catanzariti et al. (2006) Plant Cell 18:243-256). Effectors are pathogen-encoded secreted proteins that alter the functioning of host cells to facilitate infection, notably by suppressing plant immunity and controlling nutrient uptake. Immunolocalization and tissue-specific transcriptomic studies support the association of some effectors with haustoria (Dodds et al., 2004, Plant Cell 16:755-768; Kemen et al., 2005, Mol. Plant Microbe In. 18:1130-1139). These functions make haustoria formation a critical process for successful parasitic infection. In P. infestans, the glycosylated transmembrane protein PiHMP1 is required specifically for haustoria formation and critical for host colonization by P. infestans (Avrova et al., 2008, Cell. Microbiol. 10:2271-2284). Although the formation of the EHM is a key plant cell process for successful infection by many filamentous pathogens, little is known about the underlying molecular mechanisms of EHM biogenesis and function (Lu et al., 2012, Cell. Microbiol. 14:682-697; Kemen and Jones, 2012, Trends Plant Sci. 17:448-457).

Understanding how pathogens perturb host processes to promote the accommodation of infection structures such as haustoria into host cells is a major challenge in host-microbe interaction studies. The subcellular distribution of effectors inside plant cells provides valuable hints about the host cell compartments they modify to promote disease, but this approach is limited by difficulties in designing functional reporter constructs, transforming pathogen species and detecting translocated effectors in the plant cytoplasm (Whisson et al., 2007, Nature 450:115-118; Bozkurt COPB 2012). Heterologous expression of fluorescently tagged effectors in plant cells has been used to circumvent these limitations, and this approach has been used to study RXLR and Crinkler (CRN) effectors, the two major classes of cytoplasmic (host-translocated) oomycete effectors (Bozkurt et al., 2012, Curr. Opin. Plant Biol. 15:483-492). The 49 Hyaloperonospora arabidopsidis RXLR effectors studied by Caillaud et al. (Caillaud et al., 2012, Plant J. 69:252-265) localized to the nucleus, the cytoplasm or to various plant membrane compartments. In contrast, CRN effectors from several oomycete species exclusively target the plant cell nucleus (Schornack et al., 2010, PNAS 107:17421-17426). Plasma membrane localization of the Avh241 effector of P. sojae inside the plant cells is required for its cell death-inducing activity (Yu et al., 2012, New Phytol. 196:247-260). The P. infestans effectors AVRb1b2 and AVR2 accumulate around haustoria when expressed in infected N. benthamiana cells highlighting the PM and the EHM as important sites for effector activity (Bozkurt et al., 2011, PNAS 108:20832-20837; Saunders et al., 2012, Plant Cell 24:3420-3434). Animal bacterial and eukaryotic pathogens are known to secrete effectors inside the host cells that alter membrane domains to enable the assembly of perimicrobial compartments (Knodler et al., 2003, Mol. Microbiol. 49:685-704; Riglar et al., 2013, Nature Comm. 4:1415). Similarly, effectors of filamentous plant pathogens have been proposed to alter host membrane rafts to promote infection (Bhat et al., 2005, PNAS 102:3135; Caillaud et al., 2012, Plant J. 69:252-265), but whether plant membrane rafts are associated with the assembly of the host-pathogen interface and whether filamentous plant pathogen effectors target these host membrane domains is not known.

The EHM is continuous with the host plasma membrane (PM), yet it is a highly specialized membrane domain. In Arabidopsis thaliana cells infected with a powdery mildew fungus, all but one tested PM marker proteins were excluded from the EHM Koh et al., 2005, Plant J. 44:516-529; Micali et al., 2011, Cell. Microbiol. 13:210-226). Ultrastructure analysis in this interaction revealed a particular asymmetric bilayer structure for the EHM, with the inner leaflet being less electron-dense, and numerous branched invaginations around mature haustoria (Micali et al., 2011, Cell. Microbiol. 13:210-226). A survey of A. thaliana and Nicotiana benthamiana plants infected by the oomycete pathogens Hyaloperonospora arabidopsidis and P. infestans, respectively, also showed that most integral host PM proteins tested are excluded from the EHM (Lu et al., 2012, Cell. Microbiol. 14:682-697). Nevertheless, a few PM-localized proteins were reported at the EHM. The atypical Arabidopsis resistance protein RPW8.2 renders plants resistant to a broad spectrum of powdery mildew fungi (Xiao et al., 2001, Science 291:118-120) and exclusively localizes to the EHM in cells infected by Golovinomyces cichoracearum (Wang et al., 2009, Plant Cell 21:2898-2913). In the interaction of A. thaliana with Golovinomyces orontii, the syntaxin PEN1, predicted to mediate transport of extracellular defines components, accumulates in callose-containing haustorial encasements that progressively restrict the development of the haustoria in this interaction (Meyer et al., 2009, Plant J. 57:986-999). To date, there is no integral plant membrane protein reported at the EHM formed around oomycete haustoria. The StREM1.3 remorin (Raffaele et al., 2009, Plant Cell 21:1541-1555; Perraki et al., 2012, Plant Physiol. 160:624-637) and AtSYT1 Synaptotagmin (Schapire et al., 2008, Plant Cell 20:3374-3388; Yamazaki et al., 2010, J. Biol. Chem. 285:23165) peripheral membrane proteins are the only plant membrane proteins reported at the EHM in P. infestans-plant interactions (Lu et al., 2012, Cell. Microbiol. 14:682-697).

StREM1.3 belongs to a diverse family of plant specific proteins containing a Remorin_C domain (PF03763) (Raffaele et al., 2007, Plant Physiol. 145:593-600). Several proteins from the remorin family, including StREM1.3, are preferentially associated with membrane rafts, nanometric sterol- and sphingolipid-rich domains in PMs (Pike, 2006, J. Lipid Res. 47:1597; Simons and Gerl, 2010, Nature Rev. Mol. Cell Biol. 11:688-699). Indeed StREM1.3 and its tobacco homolog are highly enriched in detergent insoluble membranes (DIMs) (Shahollari et al., 2004, Physiol. Plantarum 122:397-403; Mongrand et al., 2004, J. Biol. Chem. 279:36277-36286; Raffaele et al., 2009, Plant Cell 21:1541-1555) and form sterol-dependent domains of ˜75 nm in purified PMs (Raffaele et al., 2009, Plant Cell 21:1541-1555). StREM1.3 directly binds to the cytoplasmic leaflet of the PM through a C-terminal anchor domain (RemCA) that folds into an hairpin of aliphatic alpha helices in polar environments (Raffaele et al., 2009, Plant Cell 21:1541-1555; Perraki et al., 2012, Plant Physiol. 160:624-637). StREM1.3 is differentially phosphorylated upon perception of polygalacturonic acid (Reymond et al., 1996, Plant Cell 8:2265-2276), and a related Arabidopsis protein, AtREM1.3, is differentially recruited to DIMs and differentially phosphorylated upon flg22 flagellin peptide perception (Benschop et al., 2007, Mol. Cell. Proteomics 6:1198-1214; Keinath et al., 2010, J. Biol. Chem. 285:39140; Marin et al., 2012, J. Biol. Chem. 287:39982-39991), suggesting a role in plant defense signaling. StREM1.3 and its tomato homolog SlREM1.2 prevent PVX virus spreading by interacting with TGBp1 viral movement protein, presumably in plasmodesmata or at the PM (Raffaele et al., 2009, Plant Cell 21:1541-1555; Perraki et al., 2012). AtREM1.2 belongs to protein complexes formed by the negative regulator of immune responses RPM1-INTERACTING-PROTEIN-4 (RIN4) at the PM (Liu et al., 2009, PLoS Biol. 7:e1000139). Furthermore, the Medicago truncatula MtSYMREM1 is enriched in root cells DIMs (Lefebvre et al., 2007, Plant Physiol. 144:402-418) and localizes to patches at the peribacteroid membrane during symbiosis with Sinorhizobium meliloti (Lefebvre et al., 2010, PNAS 107:2343-2348). MtSYMREM1 is important for nodule formation and interacts with the LYK3 symbiotic receptor (Lefebvre et al., 2010, PNAS 107:2343-2348). Several lines of evidence therefore implicate remorins from clade 1b (including StREM1.3) and clade 2 in cell surface signaling and accommodation during plant-microbe interactions (Raffaele et al., 2007, Plant Physiol. 145:593-600; Jarsch and Ott, 2011, Mol. Plant Microbe In. 24:7-12; Urbanus and Ott, 2012, Frontiers Plant Sci. 3:181). Nevertheless, little is known about remorin functions and their role in plant response to filamentous plant pathogens has not been reported to date.

StREM1.3 remorin is one of two plant proteins detected at the EHM during the interaction between P. infestans and N. benthamiana (Lu et al., 2012, Cell. Microbiol. 14:682-697). Therefore, studying StREM1.3 should provide useful insights into understanding the mechanisms governing the function and formation of the EHM and possibly lead to new strategies for enhancing the resistance of host plants to P. infestans and other plant pathogens that form haustoria upon the infection of a host plant.

### BRIEF SUMMARY OF THE INVENTION

Methods are provided for enhancing the resistance of plants to oomycete plant pathogens such as, for example, the economically devastating plant pathogen, Phytophthora infestans. The methods involve decreasing the level and/or activity of a remorin in the plant or part thereof. Preferably, the remorin is a remorin that is known to occur in the extrahaustorial membrane (EHM) that is formed in a host plant in response to an infection by one or more oomycete plant pathogens. The level and/or activity of such a remorin can be decreased in the host plant or part thereof for example, by disrupting in a plant cell a remorin gene that encodes the remorin, or by introducing into at least one plant cell a polynucleotide construct comprising a promoter expressible in a plant cell operably linked to a transcribed region, wherein the transcribed region is designed to produce a transcript that, when expressed in a plant cell, is capable of reducing the level of the remorin of interest in the plant cell. Such a transcribed region can comprise, for example, a nucleotide sequence that is designed for antisense-mediated gene silencing or post-transcriptional gene silencing of the remorin of interest. If desired, the plant cell can be regenerated into a transformed plant.

Methods are also provided for producing plants with enhanced resistance to one or more oomycete plant pathogens. In one embodiment, the methods comprise disrupting a remorin gene in a plant or at least one cell thereof. Such a plant with a disrupted remorin gene comprises enhanced resistance to one or more oomycete plant pathogens when compared to the resistance of a control plant that lacks the disrupted remorin gene. In another embodiment, the methods comprise stably incorporating into the genome of at least one plant cell a polynucleotide construct comprising a promoter that is expressible in a plant cell operably linked to a transcribed region as described above and regenerating the plant cell into a transformed plant comprising the polynucleotide construct. Such a transformed plant comprises enhanced resistance to one or more oomycete plant pathogens when compared to the resistance of a control plant lacking the polynucleotide construct.

Further provided are plants and plant cells comprising enhanced resistance to at least one oomycete pathogen and methods of using such plants in agricultural crop production to limit diseases caused by oomycete pathogens.

### BRIEF DESCRIPTION OF THE DRAWINGS

FIG. 1. StREM1.3 localizes at the PM and the EHM in cells infected by Phytophthora infestans. Co-expression of RFP:StREM1.3 and GFP (A), RFP:StREM1.3 and GFP:HaRXL17 (B) and YFP:StREM1.3 and RFP:AVRb1b2 (C) in haustoriated cells discriminates between host sub-cellular compartments surrounding haustoria. GFP, HaRXL17 and AVRb1b2 label the cytoplasm and the nucleus, the tonoplast, and the EHM and plasma membrane respectively. Co-localization is only observed between StREM1.3 and AVRb1b2. The fluorescence plot shows relative fluorescence along the dotted line connecting points a and b. Arrowheads point to the tip of haustoria. Bars indicate 7.5 μm. Cyt., cytoplasm; Ton. tonoplast.

FIG. 2. StREM1.3 redistributes towards the EHM during the course of Phytophthora infestans infection. (A) Frequency of haustoria surrounded by YFP:StREM1.3 in stable p35S-YFP:StREM1.3 N. benthamiana transgenic plants inoculated by P. infestans strain 88069 expressing RFP (88069td), from 2 to 5 days post inoculation (dpi) given as the percentage of all haustoria counted (n). Representative pictures taken at 3 and 5 dpi are shown with haustoria indicated by plain arrowhead when surrounded by YFP:StREM1.3 and empty arrowheads otherwise. Bars indicate 25 μm. (B) Kinetic of StREM1.3 homolog accumulation in N. benthamiana leaves sprayed with water and with P. infestans spore solution compared to untreated leaves. Histograms show anti-REM western blot signal relative to untreated sample normalized with intensity of Ponceau staining averaged from 3 independent experiments. A representative anti-REM western blot is shown. hpi, hours post inoculation. (C) P. infestans 88069td inoculated-leaves expressing YFP:StREM1.3 and stained for callose. Haustoria surrounded by YFP:StREM1.3 (plain arrowhead) never showed callose neckband (empty arrowhead).

FIG. 3. StREM1.3 co-localizes with P. infestans RXLR effector AVRb1b2 in domains at the EHM. (A) Co-expression of EHM markers in haustoriated cells reveals EHM domains. Left: YFP:StREM1.3 and RFP:AVRb1b2 show nearly full co-localization at the EHM with all domains strongly labeled by YFP:StREM1.3 (plain arrowheads) showing intense RFP fluorescence. Middle: NbSYT1 is another plant membrane protein localizing at the EHM that strongly labels domains of the EHM only weakly labeled by RFP:AVRb1b2 (empty arrowheads). Right: NbSYT1 labels domains at the EHM that are not or weakly labeled by YFP:StREM1.3 (empty arrowheads). (B) Correlation between the RFP and YFP fluorescence signals along the EHM in cells co-expressing RFP:AVRb1b2 and YFP:StREM1.3. The fluorescence is measured along the dotted line connecting points a to b as shown in the inset. The average Pearson correlation coefficient ρ for RFP and YFP fluorescence signals along 6 different EHMs is 0.79. (C) Quantification of fluorescence correlation for three EHM markers highlights the existence of at least two types of domains along the EHM. Pearson correlation coefficients ρ for the G/YFP and RFP signals were calculated in cells co-expressing NbSYT1, StREM1.3 and AVRb1b2 in different combinations (along n different EHMs). The StREM1.3-AVRb1b2 pair shows a significantly higher correlation supporting their co-localization in distinct domains at the EHM. The correlation between YFP:StREM1.3 and RFP:AVRb1b2 at two days post inoculation (dpi), when StREM1.3 does not localize at the EHM, allows to estimate the bleed through between the YFP and RFP fluorescence signals. Significance was assessed using Student t test (*, p<0.1; ***, p<0.01).

FIG. 4. Remorin silencing enhances resistance to P. infestans in N. benthamiana. (A) Validation of remorin silencing in N. benthamiana by anti-REM Western blot. Proteins were extracted from representative plants infiltrated with the pTV00 empty vector (e.v.) or with Remorin VIGS silencing construct (VIGS). (B) Type and frequency of symptoms caused by P. infestans 88069 at 5 dpi on wild type (WT), VIGS and e.v. plants as a percentage of all infection foci (total n=8 to 42). (C) Representative pictures of symptoms caused by P. infestans 88069 on N. benthamiana VIGS and e.v. plants at 7 days post inoculation (dpi). (D) Quantification of P. infestans 88069td growth in N. benthamiana lines by measure of RFP fluorescence. Representative fluorescence pictures showing P. infestans 88069td growth in VIGS and e.v. plants at 4 dpi; bars show 5 mm. Histograms show relative fluorescence intensity, calculated as the mean pixel intensity over a 0.655 cm² image centered on the lesion, and expressed as a percentage of intensity measured on WT plants. Three to 6 images were analyzed per N. benthamiana line, error bars show standard deviation.

FIG. 5. StREM1.3 overexpression increases susceptibility to P. infestans in N. benthamiana. (A) Validation of YFP:StREM1.3 over-expression in N. benthamiana transgenic plants by anti-remorin Western blot. Proteins were extracted from representative plants of p35S-YFP:StREM1.3 (OX) and wild type (WT) plants. (B) Type and frequency of symptoms caused by P. infestans 88069 at 5 days post inoculation (dpi) on OX and WT plants as a percentage of all infection foci (total n=8 to 42). (C) Representative pictures of symptoms caused by P. infestans 88069 on N. benthamiana OX and WT plants at 5 dpi. (D) Quantification of P. infestans 88069td growth in N. benthamiana lines by measure of RFP fluorescence. Representative fluorescence pictures showing P. infestans 88069td growth in OX and WT plants at 4 dpi; bars show 5 mm. Histograms show relative fluorescence intensity, calculated as the mean pixel intensity over a 0.655 cm² image centered on the lesion, and expressed as a percentage of intensity measured on WT plants. Three to 6 images were analyzed per N. benthamiana line, error bars show standard deviation. (E) N. benthamiana leaf infiltrated with Agrobacterium tumefaciens carrying either p35S-GFP or p35S-YFP:StREM1.3 (left and right half respectively) and inoculated with P. infestans 88069 24 hours later. Pictures were taken and the size of lesions measured 5 days after inoculation. (F) Relative P. infestans lesion size on half leaves infiltrated with p35S-GFP and p35S-YFP:StREM1.3. Significance was assayed using a Student t-test (p-value<0.01) over 12 lesions. (G) Total proteins extracted from half leaves infiltrated with p35S-GFP and p35S-YFP:StREM1.3 and probed by anti-GFP Western blot showing similar expression levels for GFP and YFP:StREM1.3 constructs.

FIG. 6. Remorin promotes susceptibility to P. infestans in tomato. (A) Symptoms caused by P. infestans 88069 at 4 days post inoculation (dpi) on tomato plants over-expressing the tomato StREM1.3 homolog (SE), empty vector transformed plants (e.v.), wild type (WT) and REM antisense (AS) plants. (B) Boxplot showing the distribution of the relative size of lesions at 4 dpi on tomato plants with different levels of REM. At least 48 infection foci were measure per line. Significance was assessed by a Student t-test (***p-value<0.01). Over-expression and silencing of REM was verified by Western blot on individual plants.

FIG. 7. StREM1.3 membrane binding domain is required for EHM targeting

Confocal micrographs showing the subcellular localization of YFP fusions with wild type StREM1.3, StREM1.3 lacking the C-terminal membrane anchor domain (ΔCA) and StREM1.3 with mutated C-terminal membrane anchor domain (*) in uninfected cells (A) and cells infected by P. infestans 88069td (B). The tip of haustoria is shown by a plain arrowhead when surrounded by YFP labeling, with empty arrowheads otherwise.

FIG. 8. StREM1.3 membrane anchor is required for promotion of susceptibility to P. infestans. Symptoms caused by P. infestans 88069 at 5 dpi on leaves transiently over-expressing GFP (left half of the leaf) and YFP Remorin fusions (right half of the leaf). The boxplot shows relative size of the lesions over 12 to 54 infection foci. Significance was assessed by a Student t-test (***p-value<0.01). Expression of the constructs was verified by detection of fluorescence.

FIG. 9. Phylogeny of N. benthamiana remorins. A parsimony tree of remorin proteins from A. thaliana (red), tomato (blue), potato (yellow) and N. benthamiana (green) built from a 101 amino-acids alignment of a conserved region in the Remorin_C domain. Bootstrap values for 100 replicates are indicated along the main branches. The sequence identifiers correspond to identifiers from Raffaele et al. (2007, Plant Physiol. 145:593-600) for A. thaliana remorins and Solgenomics sequence identifiers otherwise. StREM1.3 orthologs targeted by the Virus-Induced gene silencing (VIGS) construct of the present invention are indicated.

FIG. 10. Nucleotide sequence alignment of the StREM1.3 and its N. benthamiana orthologs. The StREM1.3 sequence used as a silencing construct is indicated with green line. Sites predicted to be targeted by the 21 nucleotide siRNA are shown in red.

FIG. 11. Characterization of N. benthamiana plants silenced for StREM1.3 orthologs. (A) Test for the functionality of the VIGS construct in plants constitutively expressing YFP:StREM1.3. Plants were infiltrated with the viral constructs including either the remorin silencing vector (pTV00 REM) or an empty vector (pTV00 e.v.) and observed on the day of infiltration and 18 days post-infiltration (dpi). Silencing of YFP:StREM1.3 was assessed by the loss of fluorescence, measured with identical settings. Leaves infiltrated with pTV00 REM and located 6 leaves above on infiltrated plants did not show any fluorescence at 18 dpi. (B) General aspect of plants infiltrated by pTV00 NbPDS (N. benthamiana phytoene desaturase), pTV00 Rem and pTV00 e.v. at 30 dpi. The pTV00 Rem construct did not cause any visible defect beyond what is seen on pTV00 e.v. infiltrated plants.

FIG. 12. Multiple amino acid sequence alignment used for the generation of the parsimony tree of FIG. 9.

### SEQUENCE LISTING

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth the nucleotide sequence of StREM1.3.

SEQ ID NO: 2 sets forth the amino acid sequence of StREM1.3.

SEQ ID NO: 3 sets forth the nucleotide sequence of SlREM1.2.

SEQ ID NO: 4 sets forth the amino acid sequence of SlREM1.2.

SEQ ID NO: 5 sets forth the nucleotide sequence of a sense SlREM1.2 construct.

SEQ ID NO: 6 sets forth the nucleotide sequence of an antisense SlREM1.2 construct.

SEQ ID NO: 7 sets forth the nucleotide sequence of the duplicated 35S CaMV (Cabb B-JI isolate) promoter (35SS).

SEQ ID NO: 8 sets forth the nucleotide sequence of the remorin StREM1.3 virus-induced gene silencing (VIGS) construct that is described in Example 4.

SEQ ID NO: 9 is an oligonucleotide primer that is described in Example 8.

SEQ ID NO: 10 is an oligonucleotide primer that is described in Example 8.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention is based in part on discoveries that were made during studies of the interactions between an oomycete plant pathogen, Phytophthora infestans, and a host plant. Filamentous plant pathogens such as the late blight pathogen, P. infestans, form digit-like infection structures called haustoria inside plant cells. Haustoria enable the pathogen to feed on its host, and secrete effector proteins that modulate the physiology of the host cell to facilitate infection. Haustoria are surrounded by the extrahaustorial membrane (EHM), a membrane derived from, and connected to, the plant cell plasma membrane (PM). The mechanisms controlling the formation of the EHM are almost completely unknown. Most plant membrane proteins are excluded from the EHM. As disclosed in further detail below, the StREM1.3 remorin protein is one exception as it forms domains along the EHM. The present inventors investigated the role of StREM1.3 and discovered that it enhances the susceptibility of a host plant to P. infestans. The inventors also discovered that StREM1.3 co-localizes with P. infestans effector AVRb1b2 in domains at the EHM. It is believed that StREM1.3 is the first susceptibility protein to be identified that localizes at the EHM, particularly in domains at the EHM specifically targeted by a plant pathogen effector. While the present invention is not bound by a particular biological mechanism, it is believed that P. infestans may alter plant membrane biology through one or more of its various effectors to accommodate infection structures in the host cell.

The present invention provides methods for enhancing the resistance of a plant to an oomycete plant pathogen. The methods comprise decreasing the level and/or activity of a remorin in the plant or part thereof. Preferably, the remorin is a remorin that is found in an extrahaustorial membrane (EHM) that is formed in a host plant in response to the oomycete plant pathogen. In a preferred embodiment of the invention, the remorin is the potato remorin StREM1.3 and the host plant is potato. Other remorins include, but are not limited to, potato Accession Nos. ACB28484.1 and ABU49728.1, tomato SlREM1.2 (SEQ ID NOS: 3 and 4; Accession Nos. AAD28506 and NP_001234231.1), tomato SlREM1.1 (Accession Nos. AAD28507.2 and NP_001234238.1), XP 004240737.1, and XP 004240109.1.

The methods of the present invention do not depend on a particular method for decreasing the level and/or activity of a remorin in the host plant or part thereof. Any method or methods of decreasing the level and/or activity of a protein in plant or plant cell that are known in the art or otherwise disclosed herein can be used in the methods of the present invention. Such methods include, for example, post-transcriptional gene silencing, transgenic expression of an antisense construct, and targeted mutagenesis.

In one embodiment, the method of decreasing the expression level and/or activity of remorin in a plant or part thereof comprises introducing into at least one plant cell a disruption of a remorin (REM) gene. Such a disruption decreases the expression level and/or activity of remorin in the plant cell as compared to a corresponding control plant cell lacking the disruption of the REM gene. As used herein, by “disrupt”, “disrupted” or “disruption” is understood to mean any disruption of a gene such that the disrupted gene is incapable of directing the efficient expression of a full-length fully functional gene product. The term “disrupt”, “disrupted” or “disruption” also encompasses that the disrupted gene or one of its products can be functionally inhibited or inactivated such that a gene is either not expressed or is incapable of efficiently expressing a full-length and/or fully functional gene product. Functional inhibition or inactivation can result from a structural disruption and/or interruption of expression at either the level of transcription or translation. Disruption can be achieved, for example, by at least one mutation or structural alterations, genomic disruptions (e.g. DNA insertion, DNA deletion, transposons, TILLING, homologous recombination, etc.), gene silencing elements, RNA interference, RNA silencing elements or antisense constructs. The decrease of expression and/or activity can be measured by determining the presence and/or amount of transcript (e.g. by Northern blotting or RT-PCR techniques), by determining the presence and/or amount of full-length or truncated polypeptide encoded by the disrupted gene (e.g. by ELISA or Western blotting), or by determining the presence and/or amount of remorin activity (e.g. by an activity assay or by determining oomycete pathogenicity) in the plant or part thereof with the disrupted REM gene as compared to a control plant lacking the disrupted REM gene. As used herein, it is also to be understood that “disruption” also encompasses a disruption which is effective only in a part of a plant, in a particular cell type or tissue. A disruption may be achieved by interacting with or affecting within a coding region, within a non-coding region, and/or within a regulatory region, for example, a promoter region.

For example, the activity of an remorin that is disrupted or otherwise modified by the methods disclosed herein can be assayed by determining whether the susceptibility of a host plant comprising the disrupted or modified remorin to at least one oomycete pathogen of the present invention is decreased (i.e. resistance of the plant is enhanced), when compared to the susceptibility of a control plant lacking the disrupted or modified remorin. Any method known in the art or otherwise disclosed herein can be used to assay the susceptibility of the plant to an oomycete pathogen.

Alternatively, the activity of the disrupted or modified remorin can be assayed by determining the extent to which the disrupted or modified remorin localizes to the EHM. In such an assay, localization is determined for both a host plant or host plant cells comprising the disrupted or modified remorin and a control plant or control plant cells lacking the disrupted or modified remorin. In certain embodiments of the invention, a disrupted or modified remorin with reduced activity does not localize to the EHM with the oomycte effector or localizes to a lesser extent than does the corresponding non-disrupted or non-modified remorin (e.g. a wild-type remorin). The localization of a remorin to the EMH can be determined by any method known in the art or otherwise disclosed herein such as, for example, immunochemical methods using antibodies specific to a particular remorin. Such immunochemical methods include, but are not limited to, immunofluorescence techniques involving the use of fluorophore-labeled antibodies and microscopy to detect the localization of the remorin in tissue sections comprising plant cells having an EHM.

In one embodiment the disruption of a REM gene comprises a DNA insertion. In some cases, the DNA insertion can be in the REM gene. The DNA insertion can comprise insertion of any size DNA fragment into the genome. The inserted DNA can be 1 nucleotide (nt) in length, 1-5 nt in length, 5-10 nt in length, 10-15 nt in length, 15-20 nt in length, 20-30 nt in length, 30-50 nt in length, 50-100 nt in length, 100-200 nt in length, 200-300 nt in length, 300-400 nt in length, 400-500 nt in length, 500-600 nt in length, 600-700 nt in length, 700-800 nt in length, 800-900 nt in length, 900-1000 nt in length, 1000-1500 nt in length or more such that the inserted DNA decreases the expression level and/or activity of REM. The DNA can be inserted within any region of the REM gene, including for example, exons, introns, promoter, 3′UTR or 5′UTR as long as the inserted DNA decreases the expression level and/or activity of remorin. In some embodiments the DNA can be inserted in the 5′ UTR of the REM gene, in an exon of the REM gene or in an intron of the REM gene. In specific embodiments, the DNA insertion can be in exon 1 of the REM gene, in exon 2 of the REM gene, or in intron 2 of the REM gene. The DNA to be inserted can be introduced to a plant cell by any method known in the art, for example, by using Agrobacterium-mediated recombination or biolistics. In a specific embodiment, the DNA insertion comprises a T-DNA insertion. Methods of making T-DNA insertion mutants are well known in the art.

The disruption of the REM gene can also comprise a deletion in the REM gene. As used herein, a “deletion” is understood to mean the removal of one or more nucleotides or base pairs from the DNA. Provided herein, a deletion in the REM gene can be the removal of at least 1, at least 20, at least 50, at least 100, at least 500, at least 1000, at least 5000 or more base pairs or nucleotides such that the deletion decreases the expression level and/or activity of remorin. In some cases, the entire gene can be deleted. In one embodiment, a disruption in the REM gene comprises deletion of at least one base pair from the REM gene. The DNA deletion can be within any region of the REM gene, including, for example, exons, introns, promoter, 3′ UTR or 5′UTR as long as the deletion decreases the expression level and/or activity of a remorin. The DNA deletion can be by any method known in the art, for example, by genome editing techniques as described elsewhere herein.

In some cases, the disruption of the REM gene is a homozygous disruption. By “homozygous” is understood to mean that the disruption is in both copies of the REM gene. In other cases, the disruption of the REM gene is heterozygous, that is, the disruption is only in one copy of the REM gene.

Any methods known in the art for modifying DNA in the genome of a plant can be used to alter or disrupt the coding sequences of the REM gene in planta. Such methods include genome editing techniques, such as, for example, methods involving targeted mutagenesis, homologous recombination, and mutation breeding. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for gene modification or gene replacement involving homologous recombination can involve inducing double breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Arnould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al., (2006) Nucleic Acids Res 34:e149; U.S. Pat. App. Pub. No. 2009/0133152; and U.S. Pat. App. Pub. No. 2007/0117128; all of which are herein incorporated in their entirety by reference.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

The CRISPR/Cas nuclease system can also be used to make double-strand breaks at specific recognition sequences in the genome of a plant for gene modification or gene replacement through homologous recombination. The CRISPR/Cas nuclease is an RNA-guided (simple guide RNA, sgRNA in short) DNA endonuclease system performing sequence-specific double-stranded breaks in a DNA segment homologous to the designed RNA. It is possible to design the specificity of the sequence (Cho S.W. et al., Nat. Biotechnol. 31:230-232, 2013; Cong L. et al., Science 339:819-823, 2013; Mali P. et al., Science 339:823-826, 2013; Feng Z. et al., Cell Research: 1-4, 2013).

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov F. D. et al., Nat Rev Genet. 11:636-46, 2010; Carroll D., Genetics. 188:773-82, 2011).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

Mutation breeding can also be used in the methods and compositions provided herein. Mutation breeding methods can involve, for example, exposing the plants or seeds to a mutagen, particularly a chemical mutagen such as, for example, ethyl methanesulfonate (EMS) and selecting for plants that possess a desired modification in the REM gene. However, other mutagens can be used in the methods disclosed herein including, but not limited to, radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 2500 to 2900 nm), and chemical mutagens such as base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, or acridines. Detection of such mutations typically involves high sensitivity melting curve analyses or nucleotide sequencing-based TILLING procedures. Further details of mutation breeding can be found in “Principles of Cultivar Development” Fehr, 1993 Macmillan Publishing Company the disclosure of which is incorporated herein by reference.

The methods for making modified remorin proteins that enhance resistance of a plant or plant part to an oomycete pathogen can comprise altering the coding sequence of the remorin protein, whereby the altered coding sequence encodes an amino acid sequence that comprises at least one amino acid substitution when compared to the amino acid sequence of the unmodified remorin protein. The coding sequence can be altered, for example, by making a targeted change in one or more nucleotides in the coding sequence (i.e., site directed mutagenesis) or by random mutagenesis. If desired, the altered coding sequences can then be used in assays for determining if the protein encoded thereby enhances the resistance of a plant or plant part to an oomycete pathogen. Similarly, the altered coding sequences can then be used in assays for determining if the protein encoded thereby enhances the resistance of a plant or plant part to an oomycete pathogen. The present invention does not depend on particular methods of determining whether the proteins encoded by the altered coding sequences are capable of enhancing the resistance of a plant or plant part to an oomycete pathogen. Various assays for determining resistance of a plant or plant part to an oomycete pathogen are known in the art and non-limiting examples of such assays are provided in the Example section elsewhere herein.

The methods of the present invention can comprise decreasing the expression level and/or activity of an endogenous or native REM gene in a plant or cell thereof using any method disclosed herein or otherwise known in the art. Such methods of decreasing the expression level and/or activity of a gene include, for example, in vivo targeted mutagenesis, homologous recombination, and mutation breeding. In one embodiment of the methods of the present invention, the expression of an endogenous or native REM gene is eliminated in a plant by the replacement of the endogenous or native REM gene or part thereof with a polynucleotide encoding a modified remorin protein or part thereof through a method involving homologous recombination as described elsewhere herein. In such an embodiment, the methods can further comprise selfing a heterozygous plant comprising one copy of the polynucleotide and one copy of the endogenous or native REM gene and selecting for a progeny plant that is homozygous for the polynucleotide.

Post-transcription gene silencing is the silencing or suppression of the expression of a gene that results from the mRNA of a particular gene being degraded or blocked. The degradation of the mRNA prevents translation to form an active gene product, typically a protein. The blocking of the gene occurs through the activity of silencers, which bind to repressor regions. Any method for the post-transcriptional gene silencing that is known in the art can be used in the methods of the present invention to decrease the level of one or more remorins in a plant or part thereof. Some methods of post-transcription gene silencing are further described hereinbelow including, for example, antisense suppression, sense suppression (also known as cosuppression), double-stranded RNA (dsRNA) interference, hairpin RNA (hpRNA) interference or intron-containing hairpin RNA (ihpRNA) interference, and micro RNA (miRNA) interference.

In one embodiment of the invention, the method for enhancing the resistance of a plant to an oomycete plant pathogen involves introducing a polynucleotide construct into at least one plant cell. The polynucleotide construct comprises a promoter expressible in a plant cell operably linked to a transcribed region. The transcribed region comprises a nucleotide sequence that is designed to produce a transcript for the post-transcriptional gene silencing of the remorin of interest, when the transcribed region is expressed in a plant cell. Such a transcribed region for the post-transcriptional gene silencing of the remorin is designed using any of the methods known in the art or described herein below. In general, the transcribed region will be sufficiently identical to all or to one or more fragments of the transcript of the remorin of interest and/or to the complement of the transcript produced in the plant or plant cell. While it is recognized that the degree of identity between the transcribed region and the remorin transcript or a fragment or fragments of the remorin transcript will vary depending on a number of factors such as, for example, the particular post-transcriptional gene silencing method utilized, the base composition of the nucleotide sequence of the remorin construct, and the length (i.e., number of nucleotides) of the transcribed region, a transcribed region that is sufficiently identical to all or to one or more fragments of the remorin transcript and/or complement(s) thereof will have at least about 60%, 65%, 70%, 75%, 80%, 85%, 90%, 91%, 92%, 93%, 94%, 95%, 96%, 97%, 98%, 99% or 100% sequence identity to all or to one or more fragments of the remorin transcript and/or complement(s) thereof. In one embodiment of the invention, the transcribed region will have at least about 60%, 65%, 70%, 75%, 80%, 85%, 90%, 91%, 92%, 93%, 94%, 95%, 96%, 97%, 98%, 99% or 100% sequence identity to all or to one or more fragments of the remorin transcript set forth in SEQ ID NO: 1, SEQ ID NO: 3, and/or the complements thereof.

Depending on the desired outcome, the polynucleotide constructs of the invention can be stably incorporated into the genome of the plant cell or not stably incorporated into genome of the plant cell. If, for example, the desired outcome is to produce a stably transformed plant with enhanced resistant to one or more oomycete pathogens, then the polynucleotide construct can be, for example, fused into a plant transformation vector suitable for the stable incorporation of the polynucleotide construct into the genome of the plant cell. Typically, the stably transformed plant cell will be regenerated into a transformed plant that comprises in its genome the polynucleotide construct. Such a stably transformed plant is capable of transmitting the polynucleotide construct to progeny plants in subsequent generations via sexual and/or asexual reproduction. Plant transformation vectors, methods for stably transforming plants with an introduced polynucleotide construct and methods for plant regeneration from transformed plant cells and tissues are generally known in the art for both monocotyledonous and dicotyledonous plants or described elsewhere herein.

In other embodiments of the invention in which it is not desired to stably incorporate the polynucleotide construct in the genome of the plant, transient transformation methods can be utilized to introduce the polynucleotide construct into one or more plant cells of a plant. Such transient transformation methods include, for example, viral-based methods which involve the use of viral particles or at least viral nucleic acids. Generally, such viral-based methods involve constructing a modified viral nucleic acid comprising the a polynucleotide construct of the invention operably linked to the viral nucleic acid and then contacting the plant either with a modified virus comprising the modified viral nucleic acid or with the viral nucleic acid or with the modified viral nucleic acid itself. The modified virus and/or modified viral nucleic acids can be applied to the plant or part thereof, for example, in accordance with conventional methods used in agriculture, for example, by spraying, irrigation, dusting, or the like. The modified virus and/or modified viral nucleic acids can be applied in the form of directly sprayable solutions, powders, suspensions or dispersions, emulsions, oil dispersions, pastes, dustable products, materials for spreading, or granules, by means of spraying, atomizing, dusting, spreading or pouring. It is recognized that it may be desirable to prepare formulations comprising the modified virus and/or modified viral nucleic acids before applying to the plant or part or parts thereof. Methods for making pesticidal formulations are generally known in the art or described elsewhere herein.

In one embodiment of the invention, the polynucleotide construct is operably linked to a tobacco rattle virus vector. Preferably, the tobacco rattle virus vector is pTV00 and the polynucleotide construct comprises a transcribed region designed to decrease the level of the remorin StREM1.3 in a plant or part thereof. More preferably the tobacco rattle virus vector is pTV00 and the polynucleotide construct comprises a transcribed region which comprises the nucleotide sequence set forth in SEQ ID NO: 8.

Methods are also provided for enhancing the resistance of a plant or part thereof to an oomycete pathogen by decreasing expression of an endogenous REM gene (for example, SEQ ID NOS: 10 or 24-67) in a plant by topical application of a polynucleotide molecule to the plant or part thereof. In such methods, the expression of an endogenous or native REM gene may be reduced by the introduction of ssDNA, dsDNA, ssRNA, dsRNA or RNA/DNA hybrids essentially identical to, or essentially complementary to, a sequence of 18 or more contiguous nucleotides in either the endogenous or native REM gene or messenger RNA transcribed from the REM gene through direct application with an effective amount of a transferring agent, such as, for example, an organosilicone surfactant, as described in U.S. Patent Application Publication No. 2011/0296556, hereby incorporated by reference herein in its entirety.

The methods of the present invention involve decreasing the level and/or activity of a remorin in the host plant or part thereof. While it may be desirable to decrease the level and/or activity of the remorin of interest in the entire plant, typically it will be preferred to decrease the level and/or activity of the remorin in a part or parts of the plant that are under attack or infected by the oomycete pathogen or that are likely to infected by the oomycete pathogen. Such parts include, but are not limited to, one or more of the following parts of a plant or cell thereof: leaves, stems, shoots, roots, tubers, fruits, flowers, buds, and a cell or cells within any to these plant parts. Such parts also include subcellular parts such as, for example, the EHM. In certain embodiments of the invention involving the use of a polynucleotide construct comprising a promoter expressible in plant operable linked to transcribed region, the timing and location of the decrease in the level of the remorin will be determined by the selection of the promoter. Promoters that are useful in the methods and plants disclosed herein include, but are not limited to, constitutive, tissue-preferred (e.g. leaf-preferred, root-preferred), pathogen-inducible, wound-inducible, and chemical-regulated promoters. Preferably, the promoters are pathogen-inducible and leaf-preferred promoters. More preferably, the promoters are pathogen-inducible promoters that induce gene expression in response to oomycete pathogens. Even more preferably, the promoters are pathogen-inducible promoters that induce gene expression in response to one or more oomycete pathogens in plant cells, which are at or in the vicinity of the oomycete pathogen and produce an EHM. Most preferably, the promoters are pathogen-inducible promoters that induce gene expression beginning early in the response to infection of the plant by an oomycete pathogen, and in plant cells, which are at or in the vicinity of the oomycete pathogen and produce an EHM. Such expression early in the response to infection of the plant will preferably be within about 1, 2, 3, 4, 5, 6, 7, 8, 9, 10, 12, 18, 24 hours after infection of the plant or cell thereof with the oomycete pathogen.

The methods of the present invention involve decreasing the level and/or activity of a remorin in a plant or in one or more parts thereof. Typically, the decrease in the level and/or activity of the remorin can be at least about 1%, 2%, 5%, 10%, 15%, 20%, 25%, 30%, 40%, 50%, 60%, 70%, 80%, 90% or more when compared to the level and/or activity of the remorin in a control plant or corresponding part or parts of the control plant. Generally, the control plant will be identical or nearly identical to the subject plant (i.e., the plant according one of the methods disclosed herein) and exposed to the same environmental conditions and pathogen(s) expect that the control plant will not be subjected to the method of the invention. For example, in embodiments of the invention comprising producing a subject plant that is stably transformed with a polynucleotide construct of the invention, a control plant is preferably of the same species and typically genetically identical to the subject plant except that the control plant lacks the polynucleotide construct of the invention or contains a control construct that is designed to be non-functional with respect to enhancing diseases resistance. Such a control construct might lack a promoter and/or a transcribed region or comprise a transcribed region that is unrelated to the remorin of interest.

The level or amount of remorin in plant or part thereof can be determined using standard methods known in the art including, for example, immunological methods involving the use of anti-remorin antibodies described hereinbelow.

The methods of the present invention find use in producing plants with enhanced resistance to an oomycete plant pathogen when compared to the resistance of a control plant to the oomycete plant pathogen. Typically, the methods of the present invention will enhance or increase the resistance of the subject plant to at least one oomycete pathogen by at least 25%, 50%, 100%, 150%, 200%, 250%, 500% or more when compared to the resistance of the subject plant to same one or more oomycete pathogens.

In certain embodiments of the invention, it may be desirable to decrease the level and/or activity of at least one additional remorin in the plant or part thereof. It is recognized that decreasing the level and/or activity of each additional remorin in the plant or part thereof can be accomplished essentially as described elsewhere herein for decreasing the level and/or activity of one remorin in a plant or part thereof.

The present invention further provides methods of producing a transformed plant with enhanced resistance to an oomycete plant pathogen. The methods comprising stably incorporating in the genome of at least one plant cell a polynucleotide construct of the invention as described above comprising a promoter operably linked to a transcribed region, wherein the promoter is expressible in a plant cell, and wherein the transcribed region is designed to produce a transcript for post-transcriptional gene silencing of a remorin that is found in an EHM that is formed in the plant in response to the oomycete plant pathogen. Plants produced by such methods comprise enhanced resistance to one or more oomycete plant pathogens when compared to a control plant.

Additionally, the present invention provides transformed plants, seeds, and plant cells produced by the methods of present invention and/or comprising a polynucleotide construct of the present invention. Also provided are progeny plants and seeds thereof comprising a polynucleotide construct of the present invention. The present invention also provides fruits, seeds, tubers, and other plant parts produced by the transformed plants and/or progeny plants of the invention as well as food products and other agricultural products produced from such plant parts that are intended to be consumed or used by humans and other animals including, but not limited to pets (e.g., dogs and cats) and livestock (e.g., pigs, cows, chickens, turkeys, and ducks). Other agricultural products include, for example, smoking products produced from tobacco leaves (e.g., cigarettes, cigars, and pipe and chewing tobacco) and food and industrial starch products produced from potato tubers.

The transformed plants of the present invention find use in agriculture, particularly in methods of limiting disease caused by an oomycete pathogen in agricultural crop production, the method comprising planting a transformed plant of the present invention exposing the plant to conditions favorable for growth and development of the transformed plant. Typically, the plant will be grown outdoors but alternatively can be grown in a greenhouse. The methods can further involve harvesting an agricultural product produced by the transformed plant such as, for example, a potato tuber, a tomato fruit, a pepper fruit, or a tobacco leaf.

Embodiments of the invention include, but are not limited to, the following embodiments:

1. A method for enhancing the resistance of a plant to an oomycete plant pathogen, the method comprising decreasing the level and/or activity of a remorin in the plant or part thereof, wherein the remorin is a remorin that is found in an extrahaustorial membrane (EHM) that is formed in the plant in response to the oomycete plant pathogen.

2. The method of embodiment 1, wherein the remorin is selected from the group consisting of StREM1.3 and SlREM1.2 and the remorins set forth in Accession Nos. Accession Nos. P93788, ACB28484.1, ABU49728.1, AAD28507.2, NP_001234238.1, AAD28506, NP_001234231.1, XP 004240737.1, XP 004240109.1, XP 002511833.1, XP 002510796.1, NP_001053409.1, AFK39071.1, EOX96012.1, XP 002448229.1, XP 002270914.1, CAN75437.1, XP 002267609.1, NP_001147227.1, NP_001159012.1, XP 004306803.1, XP 003580217.1, XP 003580218.1, BAJ90231.1, XP 002320784.1, XP 002322467.1, XP 002302576.1, XP 002318224.1, NP_001235181.1, NP_001236279.1, XP 003556104.1, XP 003528866.1, XP 003534573.1, XP 003521134.1, XP 004133871.1, XP 004165762.1, XP 004148376.1, EMT30253.1, XP 002878373.1, XP 002877638.1, XP 002874146.1, XP 002882035.1, NP_190463.1, NP_974824.1, NP_197764.1, AAM63910.1, NP_191685.1, NP_182106.1, AAA57124.1, AGB07445.1, AFK45936.1, AFK41243.1, XP 003638357.1, EPS60307.1, EPS59685.1, EPS69897.1, EMJ19589.1, XP 004496578.1, XP 004492710.1, EOA21483.1, EOA25547.1, EOA27951.1, XP 004976344.1, EMJ19589.1, NbS00022632g0015.1, NbS00000109g0019.1, and NbS00059367g0008.1.

3. The method of embodiment 1 or 2, wherein decreasing the level and/or activity of the a remorin in the plant or part thereof comprises introducing a polynucleotide construct into at least one plant cell, the polynucleotide comprising a promoter operably linked to a transcribed region, wherein the promoter is expressible in a plant cell, and wherein the transcribed region is designed to produce a transcript for antisense-mediated gene silencing or post-transcriptional gene silencing of the remorin.

4. The method of embodiment 3, wherein the polynucleotide construct is stably incorporated into the genome of the plant cell.

5. The method of embodiment 3 or 4, wherein the plant cell is regenerated into a plant comprising in its genome the polynucleotide construct.

6. The method of embodiment 3, the polynucleotide construct is not stably incorporated into the genome of the plant.

7. The method of embodiment 6, wherein the polynucleotide construct is in a viral vector.

8. The method of embodiment 7, wherein the viral vector is a tobacco rattle virus vector.

9. The method of embodiment 8, wherein is a tobacco rattle virus vector is pTV00.

10. The method of any one of embodiments 3-9, wherein the promoter is selected from the group consisting of pathogen-inducible, constitutive, tissue-preferred, wound-inducible, and chemical-regulated promoters.

11. The method of any one of embodiments 1-10, wherein the remorin is StREM1.3.

12. The method of embodiment 11, wherein the transcribed region comprises the nucleotide sequence set forth in SEQ ID NO: 8.

13. The method of embodiment 1 or 2, wherein decreasing the level and/or activity of the a remorin in the plant or part thereof comprises disrupting in a plant cell a remorin gene, wherein the disruption decreases the level and/or activity of the remorin in the plant cell compared to a corresponding control plant cell lacking disruption of the remorin gene.

14. The method of embodiment 13, wherein disrupting comprises an insertion, a deletion, or a substitution of a least one base pair in the remorin gene.

15. The method of embodiment 14, wherein disrupting further comprises targeted mutagenesis, homologous recombination, or mutation breeding.

16. The method of any one of embodiments 1-15, wherein the part thereof is an EHM.

17. The method of any one of embodiments 1-15, wherein the part thereof is selected from the group consisting of a leaf, a stem, a tuber, and a fruit.

18. The method of any of one of embodiments 1-15, wherein the part thereof is a plant cell.

19. The method of any one of embodiments 1-18, wherein the plant is a Solanaceous plant.

20. The method of embodiment 19, wherein the Solanaceous plant is selected from the group consisting of potato, tomato, eggplant, pepper, tobacco, and petunia.

21. The method of any one of embodiments 1-18, wherein the plant is selected from the group consisting of potato, eggplant, pepper, tobacco, petunia, lettuce, pea, bean, spinach, melon, cucumber, squash, Brassica sp., radish, onion, and watermelon.

22. The method of any one of embodiments 1-21, wherein the level and/or activity of the remorin in the plant or the part thereof is decreased when compared to the level and/or activity of the remorin in a control plant or the corresponding part of the control plant.

23. The method of any one of embodiments 1-22, wherein the plant comprises enhanced resistance to the oomycete plant pathogen when compared to the resistance of a control plant to the oomycete plant pathogen.

24. The method of any one of embodiments 1-23, further comprising decreasing the level and/or activity of at least one additional remorin in the plant or part thereof, wherein the level and/or activity of the at least one additional remorin is decreased when compared to the level and/or activity of the at least one additional remorin in a control plant.

25. The method of any one of embodiments 1-24, wherein the oomycete pathogen is selected from the group consisting of Phytophthora infestans, Phytophthora ipomoeae, Phytophthora mirabilis, Phytophthora phaseoli, Phytophthora capsici, Phytophthora porri, Phytophthora parasitica, Phytophthora ipomoeae, Phytophthora mirabilis, Hyaloperonospora arabidopsidis, Peronospora farinosa, Pseudoperonospora cubensis, Hyaloperonospora parasitica, Peronospora destructor, Bremia lactucae, Pseudoperonospora cubensis, Pseudoperonospora humuli, Peronospora destructor, Albugo candida, Albugo occidentalis, and Pythium spp.

26. A plant with enhanced resistance to an oomycete plant pathogen, the plant comprising a mutation in a remorin gene, wherein the plant has a decreased level and/or activity of remorin in the plant or part thereof as compared to a control plant that lacks enhanced resistance to the oomycte plant pathogen.

27. The plant of embodiment 26, wherein the mutation is a non-naturally occurring mutation.

28. The plant of embodiment 26 or 27, wherein the mutation comprises an insertion, a deletion, or a substitution of a least one base pair in the remorin gene.

29. The plant of any one of embodiments 26-28, wherein the plant is non-transgenic or transgenic.

30. The plant of any one of embodiments 26-29, wherein the plant is selected from the group consisting of potato, eggplant, pepper, tobacco, petunia, lettuce, pea, bean, spinach, melon, cucumber, squash, Brassica sp., radish, onion, and watermelon.

31. The plant of embodiment 30, wherein the plant is potato and the remorin is StREM1.3.

32. The plant of embodiment 30, wherein the plant is tomato and the remorin is SlREM1.2.

33. A method of producing a plant with enhanced resistance to an oomycete plant pathogen, the method comprising stably incorporating in the genome of at least one plant cell a polynucleotide construct comprising a promoter operably linked to a transcribed region, wherein the promoter is expressible in a plant cell, and wherein the transcribed region is designed to produce a transcript for antisense-mediated gene silencing or post-transcriptional gene silencing of a remorin that is found in an extrahaustorial membrane (EHM) that is formed in the plant in response to the oomycete plant pathogen.

34. The method of embodiment 33, wherein the remorin is selected from the group consisting of StREM1.3 and SlREM1.2.

35. The method of embodiment 33 or 34, wherein the plant cell is regenerated into a plant comprising in its genome the polynucleotide construct.

36. The method of any one of embodiments 33-35, wherein the level and/or activity of the remorin in the plant or part thereof is decreased when compared to the level and/or activity of the remorin in a control plant or the corresponding part of the control plant.

37. The method of any one of embodiments 33-36, wherein the plant comprises enhanced resistance to the oomycete plant pathogen when compared to the resistance of a control plant to the oomycete plant pathogen.

38. The method of embodiment 36 or 37, wherein the part thereof is an EHM.

39. The method of embodiment 36 or 37, wherein the part thereof is selected from the group consisting of a leaf, a stem, a tuber, and a fruit.

40. The method of embodiment 36 or 37, wherein the part thereof is a plant cell.

41. The method of any one of embodiments 33-40, wherein the remorin is StREM1.3.

42. The method of embodiment 41, wherein the transcribed region comprises the nucleotide sequence set forth in SEQ ID NO: 8.

43. The method of any one of embodiments 33-42, wherein the promoter is selected from the group consisting of pathogen-inducible, constitutive, tissue-preferred, wound-inducible, and chemical-regulated promoters.

44. The method of any one of embodiments 33-43, wherein the plant is a Solanaceous plant.

45. The method of embodiment 44, wherein the Solanaceous plant is selected from the group consisting of potato, tomato, eggplant, pepper, tobacco, and petunia.

46. The method of any of embodiments 33-44, wherein the plant is selected from the group consisting of potato, eggplant, pepper, tobacco, petunia, lettuce, pea, bean, spinach, melon, cucumber, squash, Brassica sp., radish, onion, and watermelon.

47. The method of any one of embodiments 33-46, further comprising stably incorporating in the genome of the at least one plant cell an additional polynucleotide construct comprising a promoter operably linked to a transcribed region, wherein the second transcribed region is designed to produce a transcript for antisense-mediated gene silencing or post-transcriptional gene silencing of a second remorin that is found in an extrahaustorial membrane (EHM) that is formed in the plant in response to the oomycete plant pathogen.

48. The method of any one of embodiments 33-47, wherein the oomycete pathogen is selected from the group consisting of Phytophthora infestans, Phytophthora ipomoeae, Phytophthora mirabilis, Phytophthora phaseoli, Phytophthora capsici, Phytophthora porri, Phytophthora parasitica, Phytophthora ipomoeae, Phytophthora mirabilis, Hyaloperonospora arabidopsidis, Peronospora farinosa, Pseudoperonospora cubensis, Hyaloperonospora parasitica, Peronospora destructor, Bremia lactucae, Pseudoperonospora cubensis, Pseudoperonospora humuli, Peronospora destructor, Albugo candida, Albugo occidentalis, and Pythium spp.

49. A transformed plant comprising stably incorporated in its genome a polynucleotide construct comprising a promoter operably linked to a transcribed region, wherein the promoter is expressible in a plant cell, and wherein the transcribed region is designed to produce a transcript for antisense-mediated gene silencing or post-transcriptional gene silencing of a remorin that is found in an extrahaustorial membrane (EHM) that is formed in the plant in response to the oomycete plant pathogen.

50. The transformed plant of embodiment 49, wherein the remorin is selected from the group consisting of StREM1.3 and SlREM1.2.

51. The transformed plant of embodiment 49 or 50, wherein the level and/or activity of the remorin in the plant or part thereof is decreased when compared to the level and/or activity of the remorin in a control plant or the corresponding part of the control plant.

52. The transformed plant of any one of embodiments 49-51, wherein the plant comprises enhanced resistance to the oomycete plant pathogen when compared to the resistance of a control plant to the oomycete plant pathogen.

53. The transformed plant of embodiment 51 or 52, wherein the part thereof is an EHM.

54. The transformed plant of any one of claim 51 or 52, wherein the part thereof is selected from the group consisting of a leaf, a stem, a tuber, and a fruit.

55. The transformed plant of any one of claim 51 or 52, wherein the part thereof is a plant cell.

56. The transformed plant of any one of claims 49-55, wherein the remorin is StREM1.3.

57. The transformed plant of any one of claims 49-56, wherein the transcribed region comprises the nucleotide sequence set forth in SEQ ID NO: 8.

58. The transformed plant of any one of claims 49-57 wherein the promoter is selected from the group consisting of pathogen-inducible, constitutive, tissue-preferred, wound-inducible, and chemical-regulated promoters.

59. The transformed plant of any one of claims 49-58, wherein the plant is a Solanaceous plant.

60. The transformed plant of embodiment 59, wherein the Solanaceous plant is selected from the group consisting of potato, tomato, eggplant, pepper, tobacco, and petunia.

61. The transformed plant of any of embodiments 49-59, wherein the plant is selected from the group consisting of potato, eggplant, pepper, tobacco, petunia, lettuce, pea, bean, spinach, melon, cucumber, squash, Brassica sp., radish, onion, and watermelon.

62. The transformed plant of any of embodiments 49-61, wherein the transformed plant is a seed or a tuber comprising the polynucleotide construct.

63. The transformed plant of any one of embodiments 49-62, wherein the oomycete pathogen is selected from the group consisting of Phytophthora infestans, Phytophthora ipomoeae, Phytophthora mirabilis, Phytophthora phaseoli, Phytophthora capsici, Phytophthora porri, Phytophthora parasitica, Phytophthora ipomoeae, Phytophthora mirabilis, Hyaloperonospora arabidopsidis, Peronospora farinosa, Pseudoperonospora cubensis, Hyaloperonospora parasitica, Peronospora destructor, Bremia lactucae, Pseudoperonospora cubensis, Pseudoperonospora humuli, Peronospora destructor, Albugo candida, Albugo occidentalis, and Pythium spp.

64. A fruit, seed, or tuber produced the plant of any one of embodiments 26-32 and 49-63.

65. A food product produced using the fruit, seed, or tuber of embodiment 64.

66. A method of limiting disease caused by an oomycete pathogen in agricultural crop production, the method comprising planting the plant according to any one of embodiments 26-32 and 49-63 and exposing the plant to conditions favorable for growth and development of the transformed plant.

67. The method of embodiment 66, wherein the plant is grown outdoors or in a greenhouse.

68. The method of embodiment 66 or 67, further comprising harvesting an agricultural product produced by the transformed plant.

69. The method of embodiment 68, wherein the product is a fruit, a leaf, or a tuber.

70. Use of the plant of any one of embodiments 26-32 and 49-63 in agriculture.

71. The use of claim 70, wherein the plant is a seed or a tuber.

Additional embodiments of the methods and compositions of the present invention are described elsewhere herein.

The methods for enhancing the resistance of a plant to one or more oomycete plant pathogens find use in increasing or enhancing the resistance of plants, particularly agricultural or crop plants, to plant pathogens. The methods of the invention can be used to enhance the resistance of any plant species including monocots and dicots. Preferred plants of the invention include Solanaceous plants, such as, for example, potato (Solanum tuberosum), tomato (Lycopersicon esculentum), eggplant (Solanum melongena), pepper (Capsicum spp.; e.g., Capsicum annuum, C. baccatum, C. chinense, C. frutescens, C. pubescens, and the like), tobacco (Nicotiana tabacum, Nicotiana benthamiana), and petunia (Petunia spp., e.g., Petunia x hybrida or Petunia hybrida). Preferred plants of the invention also include any plants that known to be infected by an oomycete pathogen including, but not limited to, P. infestans and other plant pathogenic Phytophthora species. Preferred plants of the invention that are known to be infected by an oomycete pathogen include, but are not limited to, lettuce (Lactuca sativa), pea (Pisum sativum), bean (Phaseolus vulgaris), eggplant (Solanum melongena), petunia (Petunia x hybrida), Physalis sp., woody nightshade (Solanum dulcamara), garden huckleberry (Solanum scabrum), gboma eggplant (Solanum macrocarpon), the asteraceous weeds, Ageratum conyzoides and Solanecio biafrae, palms, cocoa (Theobroma cacao), lamb's lettuce (Valerianella locusta), spinach (Spinacia oleracea), melons (including Benincasa sp., Citrullus sp., Cucumis sp., momordica sp.), cucumbers (Cucumis sp., including Cucumis sativus), Brassica sp. (including Brassica rapa), squash (Cucurbita sp.), radish (Raphanus sp.), onions (Allium sp.), cucurbits (Cucurbita sp.), hops (Humulus lupulus), watermelon (Citrullus lanatus), peach (Prunus persica), citrus trees (Citrus spp., including Citrus sinensis and Citrus clementina), Aquilegia caerulea, Malus x domestica, Linum usitatissimum, Eucalyptus grandis, cotton (Gossypium barbadense, Gossypium hirsutum, Gossypium raimondii), and Fragaria vesca. In certain embodiments, the preferred plants are all dicotyledonous plants or all dicotyledonous plants except tomato. In other embodiments, the preferred plants are all Solanaceous plants or all Solanaceous plants except tomato. In yet other embodiments, the preferred plants are potato, eggplant, pepper, tobacco, petunia, lettuce, peas, beans, spinach, melons, cucumbers, squash, Brassica sp., radish, onions, and watermelons.

Oomycete pathogens of the present invention include, but are not limited to, Phytophthora species, such as, for example, Phytophthora infestans, Phytophthora capsici, Phytophthora porri, Phytophthora parasitica, Phytophthora ipomoeae, Phytophthora mirabilis, and Phytophthora phaseoli. In other embodiments, the oomycete pathogen is Hyaloperonospora arabidopsidis, Peronospora farinosa, Pseudoperonospora cubensis, Hyaloperonospora parasitica, Peronospora destructor, Bremia lactucae, Pseudoperonospora cubensis, Pseudoperonospora humuli, Peronospora destructor, Albugo candida, Albugo occidentalis, or Pythium spp.

The polynucleotide constructs of the present invention comprise transcribed regions which can be used to reduce the expression of one or more remorins in a plant of interest. The remorins of the present invention include, but are not limited to, remorins that occur in an extrahaustorial membrane (EHM) that is formed in a plant in response to the oomycete plant pathogen. Amino acid sequences of such remorins include, for example, the amino acid sequences set forth in SEQ ID NOS: 2 and 4. Other remorins that can be used in the methods disclosed herein include those having the nucleotide or amino acid sequences set forth in Accession Nos. P93788, ACB28484.1, ABU49728.1, AAD28507.2, NP_001234238.1, AAD28506, NP_001234231.1, XP_004240737.1, XP_004240109.1, XP_002511833.1, XP_002510796.1, NP_001053409.1, AFK39071.1, EOX96012.1, XP_002448229.1, XP_002270914.1, CAN75437.1, XP_002267609.1, NP_001147227.1, NP_001159012.1, XP_004306803.1, XP_003580217.1, XP_003580218.1, BAJ90231.1, XP_002320784.1, XP_002322467.1, XP_002302576.1, XP_002318224.1, NP_001235181.1, NP_001236279.1, XP_003556104.1, XP_003528866.1, XP_003534573.1, XP_003521134.1, XP_004133871.1, XP_004165762.1, XP_004148376.1, EMT30253.1, XP_002878373.1, XP_002877638.1, XP_002874146.1, XP_002882035.1, NP_190463.1, NP_974824.1, NP_197764.1, AAM63910.1, NP_191685.1, NP_182106.1, AAA57124.1, AGB07445.1, AFK45936.1, AFK41243.1, XP_003638357.1, EPS60307.1, EPS59685.1, EPS69897.1, EMJ19589.1, XP_004496578.1, XP_004492710.1, EOA21483.1, EOA25547.1, EOA27951.1, XP_004976344.1, EMJ19589.1, NbS00022632g0015.1, NbS00000109g0019.1, and NbS00059367g0008.1; each of which is herein incorporated by reference.

The transcribed regions of the present invention are nucleotide sequences which are designed by methods disclosed herein or otherwise known in the art to silence one or more remorins that are expressed in a host plant and that are preferably known to occur in the EHM upon infection of the host plant by an oomycete pathogen of interest. Such transcribed regions are sequences that can be identical to or fully complementary to an entire native remorin polynucleotide of the present invention or a fragment thereof. Alternatively, the transcribed regions can have at least about 60%, 65%, 70%, 75%, 80%, 85%, 90%, 91%, 92%, 93%, 94%, 95%, 96%, 97%, 98%, 99% or more sequence identity to an entire native remorin polynucleotide or to a fragment thereof. In one embodiment of the invention, the transcribed regions have at least about 60%, 65%, 70%, 75%, 80%, 85%, 90%, 91%, 92%, 93%, 94%, 95%, 96%, 97%, 98%, 99% or more sequence identity to an entire remorin polynucleotide comprising the nucleotide sequence set forth in SEQ ID NO: 1 or to a fragment thereof. In another embodiment of the invention, the transcribed regions have at least about 60%, 65%, 70%, 75%, 80%, 85%, 90%, 91%, 92%, 93%, 94%, 95%, 96%, 97%, 98%, 99% or more sequence identity to an entire remorin polynucleotide comprising the nucleotide sequence set forth in SEQ ID NO: 3 or to a fragment thereof.

The present invention encompasses isolated or substantially purified polynucleotide (also referred to herein as “nucleic acid molecule”, “nucleic acid” and the like) or protein (also referred to herein as “polypeptide”) compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence protein encoded thereby. Fragments of polynucleotides comprising coding sequences may encode protein fragments that retain biological activity of the full-length or native protein. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

Polynucleotides that are fragments of a native remorin polynucleotide comprise at least 16, 20, 50, 75, 100, 125, 150, 175, 200, 250, 300, 350, 400, 450, 500, 550, or 575 contiguous nucleotides, or up to the number of nucleotides present in a full-length remorin polynucleotide disclosed herein (for example, 597 and 494 nucleotides for of SEQ ID NOS: 1 and 3, respectively). Fragments of a remorin polynucleotide useful in decreasing the level of a remorin in a plant by the methods disclosed herein generally need not encode a biologically active portion of remorin protein.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the remorins of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a remorin of the invention or can be used in decreasing the level and/or activity of a remorin in a plant by the methods disclosed herein. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptide of SEQ ID NO: 2 and 4 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Such variants may result from, for example, genetic polymorphism or from human manipulation. Biologically active variants of a remorin will have at least about 60%, 65%, 70%, 75%, 80%, 85%, 90%, 91%, 92%, 93%, 94%, 95%, 96%, 97%, 98%, 99% or more sequence identity to the amino acid sequence for the native protein (e.g. the amino acid sequence set forth in SEQ ID NO: 2 or 4) as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant and other variant forms. Likewise, the proteins of the invention encompass both naturally occurring proteins as well as variations and modified forms thereof, including, but not limited to, variations and modified forms with reduced activity. Preferably, such variants possess reduced activity, relative to the corresponding wild-type or unmodified remorin. More preferably, such variants confer to a plant or part thereof comprising the variant enhanced resistance to at least one oomycete pathogen. In some embodiments, the mutations that will be made in the DNA encoding the variant will not place the sequence out of reading frame. Optimally, the mutations will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assays that are disclosed herein below.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The polynucleotides of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that encode remorins and which hybridize under stringent conditions to at least one of the remorin polynucleotides disclosed herein or otherwise known in the art, or to variants or fragments thereof, are encompassed by the present invention.

In one embodiment, the orthologs of the present invention have coding sequences comprising at least 60%, 70%, 75%, 80%, 85%, 90%, 91%, 92%, 93%, 94%, 95%, 96%, 97%, 98%, 99%, or greater nucleotide sequence identity to a nucleotide sequence selected from the group consisting of the nucleotide sequences set forth in SEQ ID NO: 1 and SEQ ID NO: 3 and/or encode proteins comprising least 60%, 70%, 75%, 80%, 85%, 90%, 91%, 92%, 93%, 94%, 95%, 96%, 97%, 98%, 99%, or greater amino acid sequence identity to an amino acid sequence selected from the group consisting of the amino acid sequences set forth in SEQ ID NO: 2 and SEQ ID NO: 4.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the polynucleotides of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire polynucleotide disclosed herein, or one or more portions thereof, may be used as a probe capable of specifically hybridizing to corresponding polynucleotide and messenger RNAs. To achieve specific hybridization under a variety of conditions, such probes include sequences that are unique among the sequence of the gene or cDNA of interest sequences and are optimally at least about 10 nucleotides in length, and most optimally at least about 20 nucleotides in length. Such probes may be used to amplify corresponding polynucleotides for the particular gene of interest from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

It is recognized that the transcribed regions of the present invention encompass polynucleotide molecules comprising a nucleotide sequence that is sufficiently identical to the nucleotide sequence of any one or more of SEQ ID NOS: 1, 3, 5, 6, and 8. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 90%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, Md., USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website: http://www.ebi.ac.uk/Tools/clustalw/index.html).

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotide constructs comprising transcribed regions can be provided in expression cassettes for expression in the plant or other organism or non-human host cell of interest. The cassette will include 5′ and 3′ regulatory sequences operably linked to the transcribed region. “Operably linked” is intended to mean a functional linkage between two or more elements. For example, an operable linkage between a polynucleotide or gene of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the transcribed region to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a transcribed region of the invention, and a transcriptional and translational termination region (i.e., termination region) functional in plants or other organism or non-human host cell. The regulatory regions (i.e., promoters, transcriptional regulatory regions, and translational termination regions) and/or the transcribed region or of the invention may be native/analogous to the host cell or to each other. Alternatively, the regulatory regions and/or the transcribed region of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the transcribed region using heterologous promoters, the native promoter the corresponding remorin gene may be used.

The termination region may be native with the transcriptional initiation region, may be native with the operably linked transcribed region of interest, may be native with the plant host, or may be derived from another source (i.e., foreign or heterologous) to the promoter, the transcribed region of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theon. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the transcribed regions within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not intended to be limiting. Any selectable marker gene can be used in the present invention.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet.76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P:119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

The methods of the invention involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theon. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In specific embodiments, the polynucleotide constructs and expression cassettes of the invention can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91: 2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described elsewhere herein.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

The present invention may be used for transformation of any plant species, including, but not limited to, monocots and dicots. Examples of plant species of interest include, but are not limited to, peppers (Capsicum spp; e.g., Capsicum annuum, C. baccatum, C. chinense, C. frutescens, C. pubescens, and the like), tomatoes (Lycopersicon esculentum), tobacco (Nicotiana tabacum), eggplant (Solanum melongena), petunia (Petunia spp., e.g., Petunia x hybrida or Petunia hybrida), pea (Pisum sativum), bean (Phaseolus vulgaris), corn or maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), palms, oats, barley, vegetables, ornamentals, and conifers.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, tubers, propagules, leaves, flowers, branches, fruits, roots, root tips, anthers, and the like. Progeny, variants, and mutants of the regenerated plants are also included within the scope of the invention, provided that these parts comprise the introduced polynucleotides. As used herein, “progeny” and “progeny plant” comprise any subsequent generation of a plant whether resulting from sexual reproduction and/or asexual propagation, unless it is expressly stated otherwise or is apparent from the context of usage.

The methods of the present invention involve decreasing the level of a remorin in a plant or part thereof, particularly in a plant or part thereof following attack of the plant by an oomycete pathogen. In general, concentration is decreased by at least 1%, 5%, 10%, 20%, 30%, 40%, 50%, 60%, 70%, 80%, or 90% relative to a native control plant, plant part, or cell which did not have the polynucleotide construct of the invention introduced.

The expression level of the remorin may be measured directly, for example, by assaying for the level of the remorin in the plant. Methods for determining the level of a remorin include, for example, Western blot assays with anti-remorin antibodies.

In some embodiments of the present invention, a plant cell is transformed with an polynucleotide construct that is capable of expressing a polynucleotide that inhibits the expression of a remorin of interest. The term “expression” as used herein refers to the biosynthesis of a gene product, including the transcription and/or translation of said gene product. For example, for the purposes of the present invention, polynucleotide construct capable of expressing a transcribed region that inhibits the expression of at least one remorin in a host plant of interest is a polynucleotide construct capable of producing an RNA molecule that inhibits the transcription and/or translation of at least one remorin in the host plant. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide. Examples of polynucleotides that inhibit the expression of a remorin are provided below.

In some embodiments of the invention, inhibition of the expression of a remorin may be obtained by sense suppression or cosuppression. For cosuppression, an expression cassette is designed to express an RNA molecule corresponding to all or part of a messenger RNA encoding a remorin in the “sense” orientation. Overexpression of the RNA molecule can result in reduced expression of the native gene. Accordingly, multiple plant lines transformed with the cosuppression expression cassette are screened to identify those that show the greatest inhibition of remorin expression.

The polynucleotide used for cosuppression may correspond to all or part of the sequence encoding the remorin, all or part of the 5′ and/or 3′ untranslated region of a remorin transcript, or all or part of both the coding sequence and the untranslated regions of a transcript encoding a remorin. In some embodiments where the polynucleotide comprises all or part of the coding region for the remorin, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin et al. (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell et al. (1994) Proc. Natl. Acad. Sci. USA 91:3490-3496; Jorgensen et al. (1996) Plant Mol. Biol. 31:957-973; Johansen and Carrington (2001) Plant Physiol. 126:930-938; Broin et al. (2002) Plant Cell 14:1417-1432; Stoutjesdijk et al (2002) Plant Physiol. 129:1723-1731; Yu et al. (2003) Phytochemistry 63:753-763; and U.S. Pat. Nos. 5,034,323, 5,283,184, and 5,942,657; each of which is herein incorporated by reference. The efficiency of cosuppression may be increased by including a poly-dT region in the expression cassette at a position 3′ to the sense sequence and 5′ of the polyadenylation signal. See, U.S. Patent Publication No. 20020048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

In some embodiments of the invention, inhibition of the expression of the remorin may be obtained by antisense suppression. For antisense suppression, the expression cassette is designed to express an RNA molecule complementary to all or part of a messenger RNA encoding the remorin. Overexpression of the antisense RNA molecule can result in reduced expression of the native gene. Accordingly, multiple plant lines transformed with the antisense suppression expression cassette are screened to identify those that show the greatest inhibition of remorin expression.

The polynucleotide for use in antisense suppression may correspond to all or part of the complement of the sequence encoding the remorin, all or part of the complement of the 5′ and/or 3′ untranslated region of the remorin transcript, or all or part of the complement of both the coding sequence and the untranslated regions of a transcript encoding the remorin. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used. Methods for using antisense suppression to inhibit the expression of endogenous genes in plants are described, for example, in Liu et al (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, U.S. Patent Publication No. 20020048814, herein incorporated by reference.

In certain embodiments of the invention, a full-length remorin transcript is used for antisense and sense suppression as disclosed hereinbelow for remorin-silenced tomatoes. In other embodiments, the specificity of silencing can be achieved by designing antisense constructs based on non-conserved sequence regions of a remorin nucleotide sequence, which could correspond to the region encoding the N-terminal domain for a remorin. Alternatively, longer antisense constructs can be used that would preferentially form and RNA duplex with the closest endogenous RNA. This later strategy was used to generate remorin-silenced tomatoes disclosed hereinbelow, because the diversity of remorin family in tomato was not known when the silencing construct was tested. In other embodiments, several remorins can be silenced with a single antisense or sense construct that is designed based on the conserved remorin C-terminal domain.

In some embodiments of the invention, inhibition of the expression of a remorin may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of remorin expression. Methods for using dsRNA interference to inhibit the expression of endogenous plant genes are described in Waterhouse et al. (1998) Proc. Natl. Acad. Sci. USA 95:13959-13964, Liu et al. (2002) Plant Physiol. 129:1732-1743, and WO 99/49029, WO 99/53050, WO 99/61631, and WO 00/49035; each of which is herein incorporated by reference.

In some embodiments of the invention, inhibition of the expression of one or more remorins may be obtained by hairpin RNA (hpRNA) interference or intron-containing hairpin RNA (ihpRNA) interference. These methods are highly efficient at inhibiting the expression of endogenous genes. See, Waterhouse and Helliwell (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:4985-4990; Stoutjesdijk et al. (2002) Plant Physiol. 129:1723-1731; and Waterhouse and Helliwell (2003) Nat. Rev. Genet. 4:29-38. Methods for using hpRNA interference to inhibit or silence the expression of genes are described, for example, in Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407:319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407:319-320; Wesley et al. (2001) Plant J. 27:581-590; Wang and Waterhouse (2001) Curr. Opin. Plant Biol. 5:146-150; Waterhouse and Helliwell (2003) Nat. Rev. Genet. 4:29-38; Helliwell and Waterhouse (2003) Methods 30:289-295, and U.S. Patent Publication No. 20030180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904, herein incorporated by reference.

Transcriptional gene silencing (TGS) may be accomplished through use of hpRNA constructs wherein the inverted repeat of the hairpin shares sequence identity with the promoter region of a gene to be silenced. Processing of the hpRNA into short RNAs which can interact with the homologous promoter region may trigger degradation or methylation to result in silencing (Aufsatz et al. (2002) PNAS 99 (Suppl. 4):16499-16506; Mette et al. (2000) EMBO J 19(19):5194-5201).

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for a remorin). Methods of using amplicons to inhibit the expression of endogenous plant genes are described, for example, in Angell and Baulcombe (1997) EMBO J. 16:3675-3684, Angell and Baulcombe (1999) Plant J. 20:357-362, and U.S. Pat. No. 6,646,805, each of which is herein incorporated by reference.

In some embodiments, the polynucleotide expressed by the expression cassette of the invention is catalytic RNA or has ribozyme activity specific for the messenger RNA of a remorin. Thus, the polynucleotide causes the degradation of the endogenous messenger RNA, resulting in reduced expression of the remorin. This method is described, for example, in U.S. Pat. No. 4,987,071, herein incorporated by reference.

In some embodiments of the invention, inhibition of the expression of one or more remorins may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier et al. (2003) Nature 425: 257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of remorin expression, the 22-nucleotide sequence is selected from a remorin transcript sequence and contains 22 nucleotides of said sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

The use of the terms “DNA” or “RNA” herein is not intended to limit the present invention to polynucleotide molecules comprising DNA or RNA. Those of ordinary skill in the art will recognize that the methods and compositions of the invention encompass polynucleotide molecules comprised of deoxyribonucleotides (i.e., DNA), ribonucleotides (i.e., RNA) or combinations of ribonucleotides and deoxyribonucleotides. Such deoxyribonucleotides and ribonucleotides include both naturally occurring molecules and synthetic analogues including, but not limited to, nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The polynucleotide molecules of the invention also encompass all forms of polynucleotide molecules including, but not limited to, single-stranded forms, double-stranded forms, hairpins, stem-and-loop structures, and the like. Furthermore, it is understood by those of ordinary skill in the art that the nucleotide sequences disclosed herein also encompasses the complement of that exemplified nucleotide sequence.

The invention is drawn to compositions and methods for enhancing the resistance of a plant to plant disease. By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen is minimized or lessened.

The following examples are offered by way of illustration and not by way of limitation.

### Examples

To gain insights on the biogenesis and function of the EHM, the role of StREM1.3 remorin was investigated. StREM1.3 remorin is one of the few plant membrane proteins to accumulate at the EHM during infection of N. benthamiana by P. infestans (Lu 2012). The results are disclosed in the following examples and subsequent discussion section.

### Example 1: StREM1.3 Localizes at the PM and the EHM in Cells Infected by Phytophthora infestans

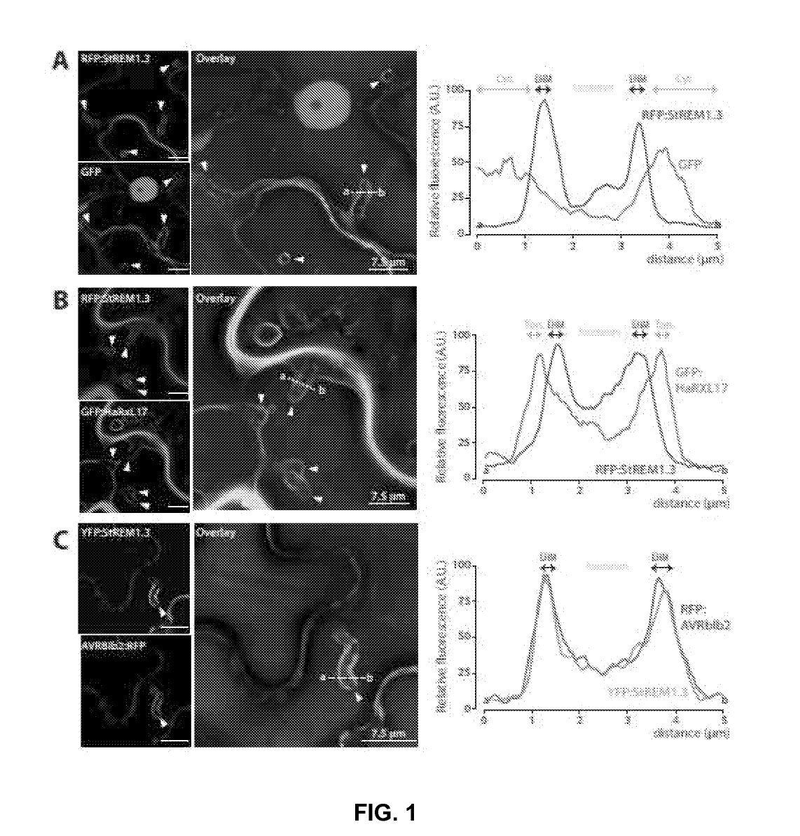

N. benthamiana is a versatile host system to study the cellular and molecular dynamics of the plant response to the hemibiotrophic pathogen P. infestans (Chaparro-Garcia et al., 2011, PLoS One 6:e16608; Lu et al., 2012, Cell. Microbiol. 14:682-697). Using fluorescent markers for the cytoplasm, tonoplast and EHM, it was found that these three subcellular compartments occur closely around P. infestans haustoria (Caillaud et al., 2012, Plant J. 69:252-265; Bozkurt et al., 2012, Curr. Opin. Plant Biol. 15:483-492), which makes it challenging to distinguish EHM from these other compartments. To determine in which perihaustorial compartment StREM1.3 resides, we performed a series of co-localization studies using various marker proteins labeling distinct perihaustorial compartments in N. benthamiana plants inoculated by P. infestans. First, to determine whether StREM1.3 actually localizes to the EHM or remains in the cytoplasm surrounding them, we co-expressed RFP:StREM1.3 and GFP in infected plant cells . . . . After four days post inoculation (dpi), RFP:StREM1.3 fluorescence surrounded P. infestans haustoria tightly, and showed a sharp and focused signal in contrast to the diffuse cytosolic localization pattern of GFP suggesting that StREM1.3 localizes at the EHM (FIG. 1A). Second, to exclude the possibility that REM1.3 accumulates at the tonoplast surrounding EHM, we co-expressed RFP:StREM1.3 with H. arabidopsidis effector HaRXL17, which marks the perihaustorial tonoplast in host cells surrounding P. infestans haustoria (Caillaud et al., 2012, Plant J. 69:252-265; Bozkurt et al., 2012, Curr. Opin. Plant Biol. 15:483-492). After 4 dpi, RFP:StREM1.3 fluorescence was tightly surrounded by GFP:HaRXL17 fluorescence. In plots measuring fluorescence along a line cutting through haustoria, the two peaks of RFP:StREM1.3 fluorescence were located between the two peaks of GFP:HaRXLraff17, indicating that StREM1.3 localizes between the tonoplast and the haustorium. Finally, we co-expressed YFP:StREM1.3 with P. infestans RXLR effector AVRb1b2 that accumulates at the EHM in infected plant cells (Bozkurt et al., 2011, PNAS 108:20832-20837; Bozkurt et al., 2012, Curr. Opin. Plant Biol. 15:483-492) (FIG. 1C). Remarkably, StREM1.3 and AVRb1b2 co-localized almost completely around haustoria clarifying that StREM1.3 indeed traffics to the EHM.

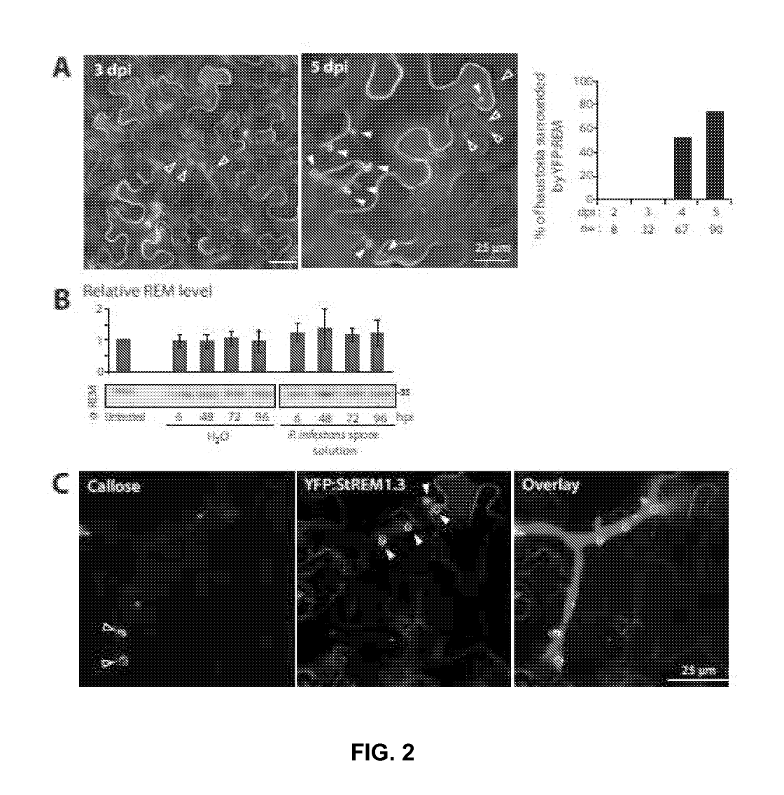

Unlike the powdery mildew pathogen or Hpa, P. infestans haustoria are rarely surrounded by callose encasements but sometimes may accumulate a callosic neck band (Bozkurt et al., 2011, PNAS 108:20832-20837). Callose encasements are thought to be indicative of a plant defense reaction and do not reflect a viable haustorial interface (van van Damme et al., 2009, Plant Cell 21:2179-2189). To determine the degree to which StREM1.3 perihaustorial accumulation is associated with callose, we performed aniline blue staining on plants expressing YFP:StREM1.3 and infected by P. infestans strain (88069td) expressing a cytosolic RFP. We found that StREM1.3-labelled haustoria do not display a callosic neck band (FIG. 2C) indicating that StREM1.3-labelled haustoria are active infection structures and that the perihaustorial localization of StREM1.3 is not a result of membrane encasement of the haustoria.

### Example 2: StREM1.3 Redistributes to the EHM of Viable Haustoria During Plant Infection by P. infestans

To document the dynamics of StREM1.3 accumulation around P. infestans haustoria, we used N. benthamiana transgenic plants constitutively expressing YFP:StREM1.3 (Lu et al., 2012, Cell. Microbiol. 14:682-697). We inoculated these plants with a transgenic P. infestans isolate 88069 constitutively expressing the red fluorescent marker tandem dimer RFP (88069td) (Chaparro-Garcia et al., 2011, PLoS One 6:e16608), and monitored the distribution of YFP:StREM1.3 in infected cells over time. Surprisingly, up to three days post inoculation (dpi) we observed a decrease in fluorescence at the PM in proximity to haustoria (FIG. 2A). The PM of infected cells appeared strongly polarized with areas distal to haustoria retaining YFP fluorescence. Surprisingly, we found no haustoria surrounded by YFP:StREM1.3 at 2 and 3 dpi. Interestingly, from 4 dpi, polarization of the PM disappeared resulting in distribution of YFP:StREM1.3 fluorescence all along the PM and the EHM (FIG. 2A). YFP:StREM1.3 signal around haustoria appeared very intense and frequently organized as foci. Approximately 50% of haustoria were surrounded by YFP:StREM1.3 at 4 dpi, which increased to ˜80% at 5 dpi (FIG. 1A).

To test whether the decrease of YFP:StREM1.3 fluorescence around the -pathogen contact sites at the early stages of P. infestans infection is due to altered protein turnover in infected cells, we measured the total level of endogenous StREM1.3 homolog in N. benthamiana through a time course of infection. For this, we used antibodies directed to the full length StREM1.3 protein, taking advantage of the high conservation between remorins in the Solanaceae (Raffaele et al., 2009, Plant Cell 21:1541-1555). The overall level of remorin protein remained constant during infection with only a slight (˜1.2 fold in average) increased accumulation around 48 hpi (FIG. 2B). No significant decrease in remorin level could be detected in these experiments indicating that the remorin pool remains constant but redistributes towards the EHM during infection by P. infestans.

### Example 3: StREM1.3 Co-Localizes with P. infestans RXLR Effector AVRblb2 in Specific Domains at the EHM

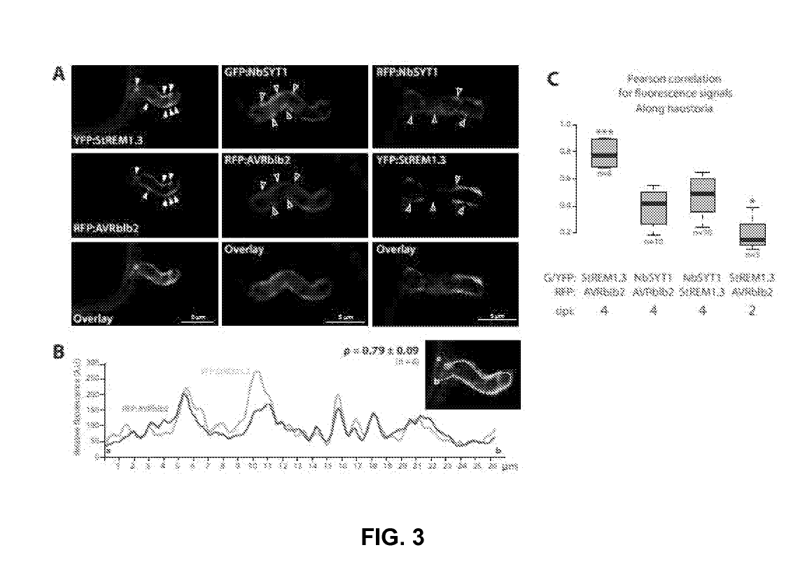

StREM1.3 is a well-established protein marker of sterol- and sphingolipid-rich PM domains designated as membrane rafts (Raffaele et al., 2009, Plant Cell 21:1541-1555). We observed that StREM1.3 displays non uniform perihaustorial accumulation, delimiting discrete membrane domains at the EHM (FIG. 1). Similar to Rem1.3, the P. infestans RXLR effector AVRblb2 localizes to the PM and dramatically re-localizes to the EHM during host infection (Bozkurt et al., 2011, PNAS 108:20832-20837; Bozkurt et al., 2012, Curr. Opin. Plant Biol. 15:483-492). We observed that from 4 dpi, StREM1.3 and AVRblb2 significantly co-localize at the EHM in haustoria cross-sections (FIG. 1). To test whether AVRblb2 specifically targets StREM1.3-containing membrane domains, we examined the degree to which these two proteins co-localize in haustoria longitudinal sections. For this, we co-expressed YFP:StREM1.3 and RFP:AVRb1b2 in N. benthamiana infected cells at 4 dpi. YFP:StREM1.3 localized to foci along the EHM, that frequently accumulated at the neck and toward the tip of haustoria, and more rarely associated with the base of haustoria (FIG. 3A, arrowheads). RFP:AVRb1b2 also showed variations along the EHM with more intense foci co-localizing with YFP:StREM1.3 foci. To quantify the degree of StREM1.3 and AVRblb2 co-localization, we extracted fluorescence signals along the EHM and calculated Pearson correlation coefficients (p, FIG. 3B). Average Pearson correlation coefficient between the YFP and RFP fluorescence signal along the EHM was 0.79 indicating that StREM1.3 and AVRblb2 target the same domains along the EHM.

To further define the membrane domains we highlighted at the EHM, we co-expressed StREM1.3 and AVRblb2 with SYT1, another plant protein localized at the EHM (Lu et al., 2012, Cell. Microbiol. 14:682-697). SYT1 localized in foci along the EHM that mostly accumulated at the base of haustoria, as opposed to AVRblb2 and StREM1.3 (FIG. 3A). We calculated pairwise correlation coefficient for the fluorescence associated with YFP:StREM1.3, RFP:AVRb1b2 and G/RFP:SYT1 along 6 to 10 different EHM confocal images (FIG. 3C). As mentioned above, YFP:StREM1.3 and RFP:AVRb1b2 almost fully overlapped with an average ρ˜0.8. By contrast, average correlation between GFP:SYT1 and RFP:AVRb1b2, and RFP:SYT1 and YFP:StREM1.3, yielded ρ˜0.4 and 0.5 respectively. To estimate the background correlation associated with the bleed through of YFP and RFP fluorescence signals in our experimental conditions, we calculated ρ for YFP:StREM1.3 and RFP:AVRb1b2 along the EHM at 2 dpi, when the two proteins do not co-localize. As expected, an average ρ<0.2 was found in that case. These results show that the EHM exhibit lateral compartmentalization with StREM1.3 and AVRb1b2 targeting the same domains. The assembly of these domains appeared dynamic with AVRb1b2 accumulation preceding that of StREM1.3.

### Example 4: Remorin Silencing Enhances Resistance to P. infestans in N. benthamiana

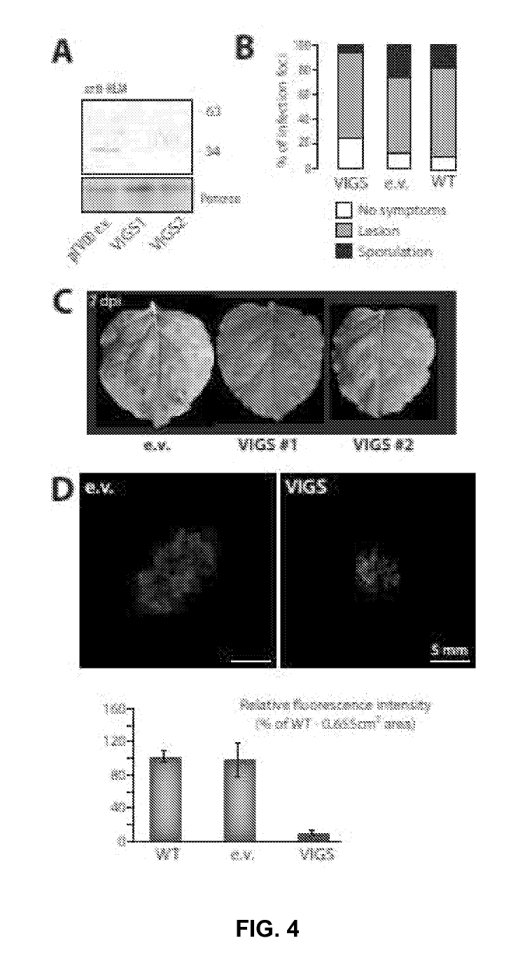

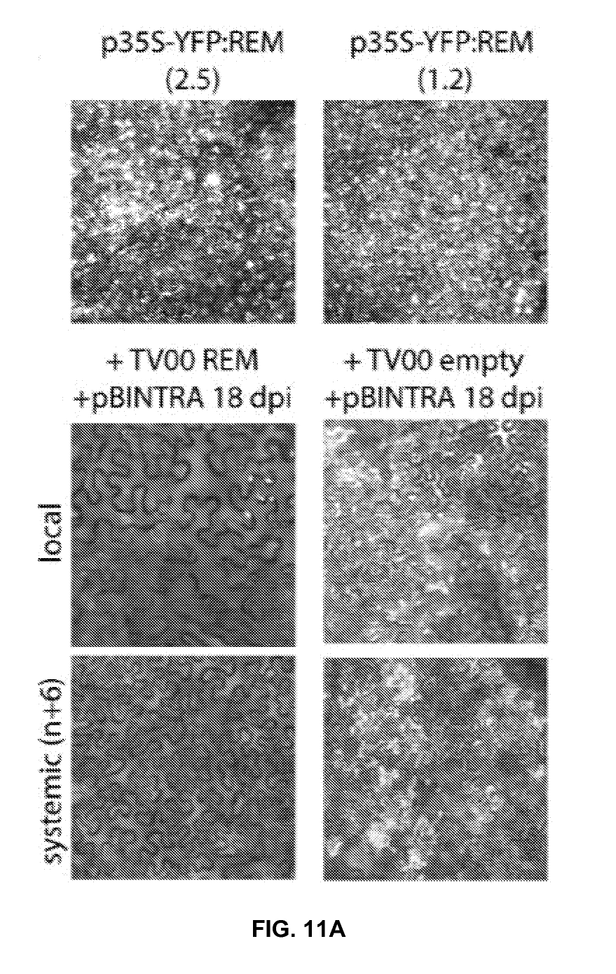

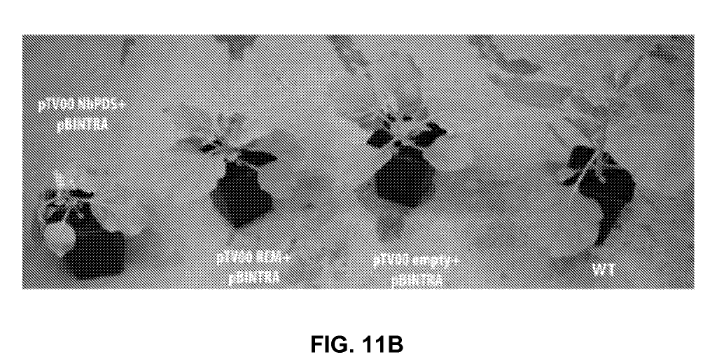

Localization of StREM1.3 at the plant-pathogen interface prompted us to test whether REM1.3 plays a role in immunity against P. infestans. For this, we performed a virus-induced gene silencing (VIGS) approach to silence the StREM1.3 ortholog in N. benthamiana using the Tobacco rattle virus (TRV) pTV00 vector (Ratcliff 2001). Eighteen days after delivery of the StREM1.3 silencing construct but not with the empty vector control (pTV00) we observed a strong decrease in YFP fluorescence in N. benthamiana plants stably expressing YFP:StREM1.3-validating the efficiency of silencing (FIG. 11). In addition, an anti-Remorin Western Blot performed on total protein extracts from wild-type and silenced (VIGS) N. benthamiana plants confirmed the suppression of remorin accumulation by our silencing construct (FIG. 4A). Six-week old plants silenced for remorin did not show any apparent developmental phenotype (FIG. 11). We then tested the response of remorin-silenced plants to P. Infestans using spore solution droplet inoculation. At 5 dpi, ˜20% of infection foci on wild type (WT) plants and control plants expressing the pTV00 empty vector (e.v.) showed sporulation, whereas this proportion was <5% for foci on remorin-silenced plants (FIG. 4B). Conversely, although 20% of infection foci on remorin-silenced did not show any symptom, this proportion was reduced to <10% on WT and e.v. plants. At 7 dpi, confluent lesions caused by P. infestans growth were clearly visible on control plants expressing the pTV00 empty vector, whereas the lesions hardly extended beyond the spore droplets in remorin-silenced plants (FIG. 4C). To confirm that lesion size correlates with pathogen growth in these plants, we used image analysis to quantify the surface occupied by hyphae of P. Infestans 88069td. We measured a ˜10 fold decrease in the surface colonized by P. infestans 88069td in remorin-silenced plants compared to WT and e.v. plants (FIG. 4D).

### Example 5: StREM1.3 Overexpression Increases Susceptibility to P. infestans in N. benthamiana

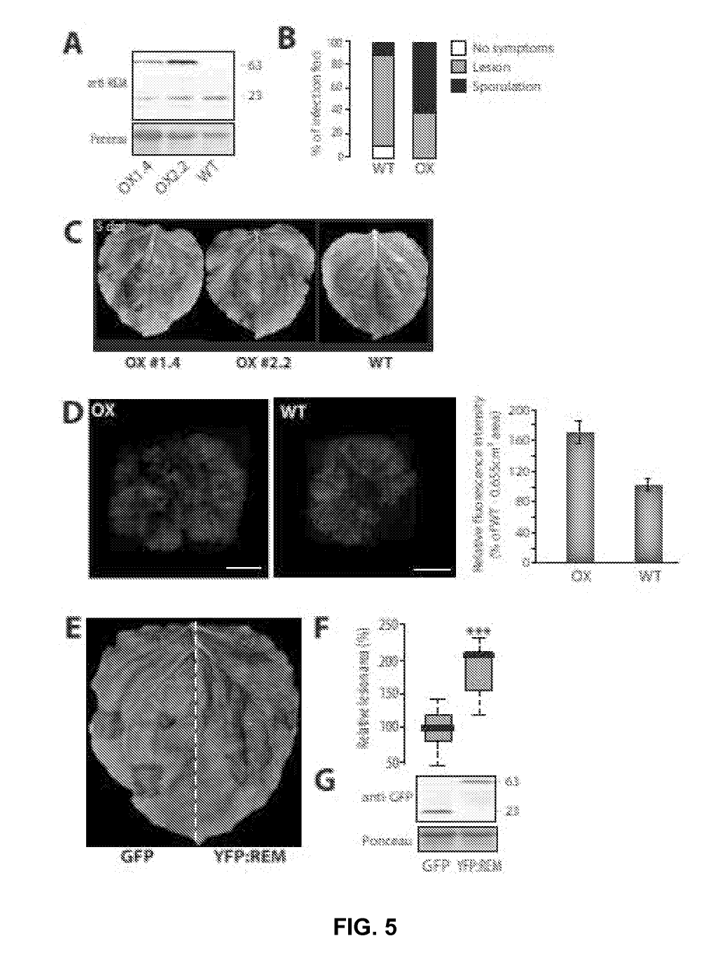

To further characterize the role of StREM1.3 in response to P. infestans, we analyzed the phenotype of transgenic plants constitutively expressing YFP:StREM1.3 (OX). We first verified the expression and integrity of the YFP:StREM1.3 fusion protein in these plants using anti-remorin Western blot (FIG. 5A). We next tested the response of these plants to P. Infestans using spore solution droplet inoculation. We counted the proportion of inoculated sites showing no symptom, necrotic lesion or P. infestans sporulation on WT and OX plants. We found that the frequency of P. Infestans sporulation correlated with higher REM accumulation, whereas the frequency of inoculated areas with no symptom correlated with reduced REM accumulation (FIG. 5B). At 5 dpi, lesions caused by P. infestans growth almost completely covered OX plant leaves, whereas the lesions hardly extended beyond the spore droplets in WT plants (FIG. 5C). Using image analysis to quantify the surface occupied by hyphae of P. Infestans 88069td we found a ˜1.5 fold increase in OX plants compared to WT (FIG. 5D). We also used transient Agrobacterium-mediated over-expression of YFP:StREM1.3 in N. benthamiana. In this assay, one half of the leaf was infiltrated with an A. tumefaciens carrying the p35S-GFP construct as a control, and the other half with a strain carrying the p35S-YFP:StREM1.3 construct. The leaves were inoculated by P. infestans spore solution 24 hours later, and the infected area measured 5 days after inoculation. In half leaves over-expressing REM, the infected area was in average twice as large as in half leaves over-expressing GFP (FIG. 5E, F) indicating that REM over-expression enhanced susceptibility to P. Infestans. Quantification of the fluorescence due to the GFP and YFP expression as well as anti-GFP western blots performed on total protein extracts allowed to select for leaves in which the two Agrobacterium-delivered constructs were expressed to similar levels (FIG. 5G). Taken together, these results indicate a negative role for StREM1.3 in immunity against P. infestans.

### Example 6: Remorin Promotes Susceptibility to P. infestans in Tomato

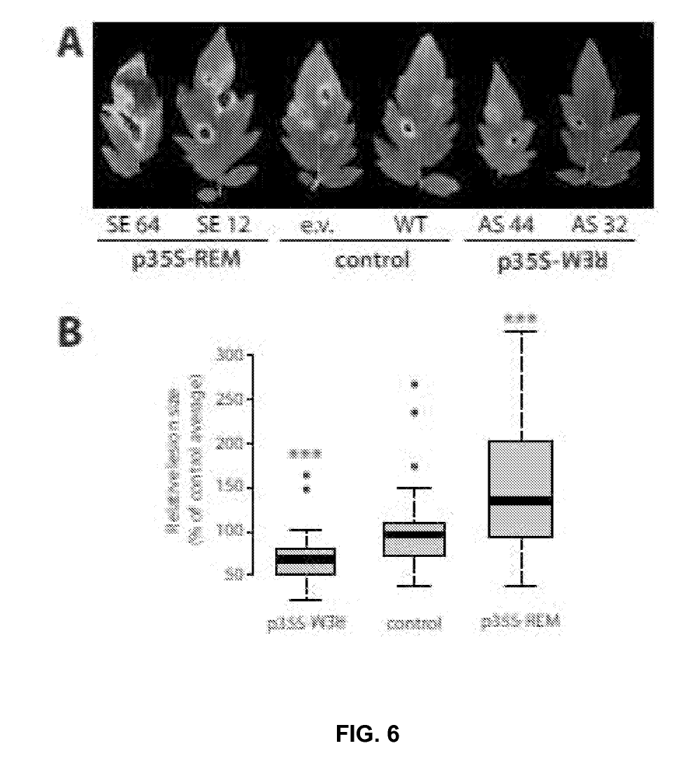

Most cultivated plants in the Solanaceae family, including tomato and potato, are susceptible to P. Infestans. To test whether the function of remorin in N. benthamiana response to P. infestans is conserved in economically important crops, we inoculated tomato transgenic plants expressing sense and antisense SlREM1.2 constructs, the closest homolog of StREM1.3 in tomato, with spore solutions of P. infestans (Raffaele et al., 2009, Plant Cell 21:1541-1555). The level of SlREM1.2 in individual plants was evaluated by anti-Remorin Western Blot prior to infection to 21-87% and 107-251% of wild type in antisense and sense lines respectively. In plants over-expressing REM (SE), P. infestans-induced lesions appeared significantly larger than in wild type and control plants (150% of wild type in average and up to 300%, FIG. 6). Conversely, plants expressing an antisense REM construct showed reduced lesions (75% of wild type in average). Statistics calculated on ˜50 infection foci per line supported the conclusion that REM promotes susceptibility to P. infestans in tomato. We observed similar degree of increase in P. infestans infection in N. benthamiana plants overexpressing YFP:StREM1.3 and in tomato plants overexpressing untagged SlREM1.2, indicating that StREM1.3 and SlREM1.2 homologs have similar function in response to P. infestans, the YFP tag does not significantly alter this function, and the molecular mechanisms underlying this function are conserved in N. benthamiana and tomato.

### Example 7: StREM1.3 Membrane Anchor is Required for Re-Localization at the EHM

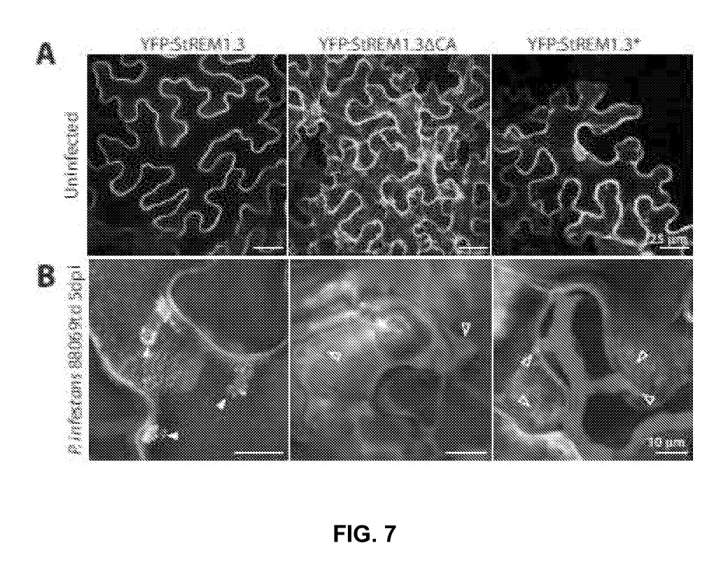

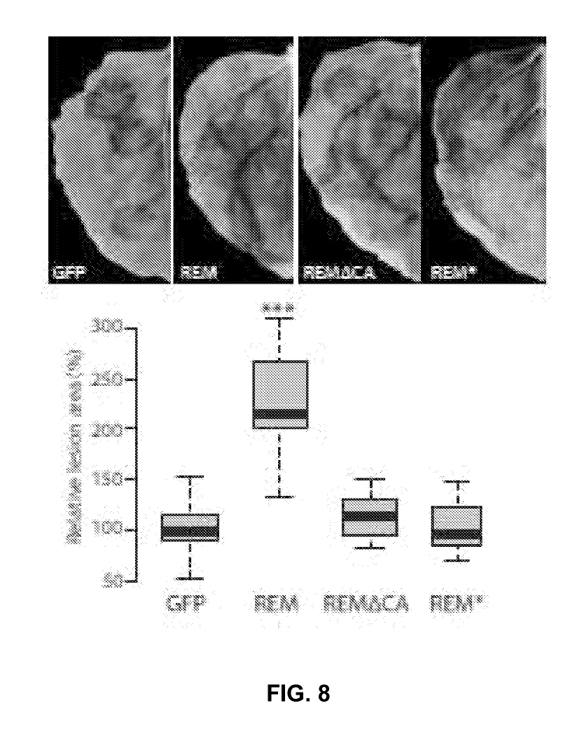

We recently demonstrated that StREM1.3 is targeted to the plasma membrane (PM) through direct lipid binding of a C-terminal alpha helical domain named Remorin C-terminal Anchor (RemCA) (Perraki et al., 2012, Plant Physiol. 160:624-637). To test whether StREM1.3 PM binding is also required for re-localization to the EHM, we expressed YFP-tagged wild type and mutant StREM1.3 constructs in N. benthamiana using Agrobacterium-mediated transformation. Consistent with previous reports, YFP:StREM1.3 localized exclusively at the PM whereas mutants lacking the RemCA domain (YFP:StREM1.3ΔCA) or mutated in the RemCA domain (YFP:StREM1.3*) localized to the cytoplasm in non-infected N. benthamiana epidermal cells (FIG. 7A). We subsequently inoculated transformed leaves with P. infestans 88069td and observed haustoria formed in transformed cells at 4 and 5 dpi. As reported earlier, a strong YFP accumulation is visible around approximately ˜70% of haustoria formed in YFP:StREM1.3-expressing cells. By contrast, a uniform cytoplasmic YFP localization is seen in YFP:StREM1.3ΔCA and YFP:StREM1.3*-expressing cells, none of the haustoria observed in these cells showed accumulation of YFP fluorescence (>30 haustoria surveyed for each construct, FIG. 7B). The RemCA membrane anchor is therefore required for StREM1.3 re-localization at P. infestans EHM.

### Example 8: StREM1.3 Membrane Anchor is Required for Promotion of Susceptibility to P. infestans

To test whether StREM1.3 PM binding is required for promotion of susceptibility to P. infestans, we measured P. infestans lesion size formed on N. benthamiana leaves expressing full length or mutated StREM1.3 constructs. Half leaves expressing YFP:StREM1.3 showed lesions ˜250% the size of half leaves expressing GFP control, whereas sectors expressing YFP:StREM1.3ΔCA or YFP:StREM1.3* showed lesions the same size as in half leaves infiltrated with the GFP control (FIG. 8). These results indicate that PM anchoring is required for StREM1.3 function in response to P. infestans. Results obtained with StREM1.3 mutants therefore connect StREM1.3 PM anchoring, re-distribution towards the EHM and promotion of susceptibility to P. infestans.

### DISCUSSION

A combination of cell biology and pathology assays were used to document the redistribution of StREM1.3 towards discrete domains at the EHM that are also targeted by the RXLR effector AVRb1b2. Genetic analyses revealed that remorin promotes susceptibility to P. infestans, and can therefore be considered a susceptibility factor (Vogel et al., 2002, Plant Cell 14:2095-2106). Thus StREM1.3 is the first plant susceptibility protein shown to localize at the EHM, supporting the view that haustoria-forming plant pathogens interfere with the membrane biogenesis machinery of their host to promote intracellular accommodation inside host cells and infection.

Although many plant PM proteins are excluded from the EHM, StREM1.3 localized to discrete domains within the EHM. StREM1.3 is a well-established plant membrane raft marker protein that binds directly to negatively-charged lipids enriched in plant membrane rafts (Raffaele et al., 2009, Plant Cell 21:1541-1555; Furt et al., 2010, Plant Physiol. 152:2173-2187; Perraki 2012). The redeployment of StREM1.3 at the EHM suggests that this membrane may have a lipid composition close to that of membrane rafts. Kemen et al. (Kemen and Jones, 2012, Trends Plant Sci. 17:448-457) showed that, in A. thaliana, the EHM surrounding the haustoria of another oomycete pathogen Albugo laibachii is rich in sterols or sterol-like molecules, a typical feature of detergent insoluble membranes and membrane rafts (Cacas et al., 2012, Prog. in Lipid Res. 51:272-299; Simon-Plas et al., 2011, Curr. Opin. Plant Biol. 14:642-649). Similarly, the periarbuscular and peribactoid membranes formed during fungal and bacterial plant endosymbiosis, also share similarities with membrane rafts (Pumplin and Harrison, 2009, Plant Physiol. 151:809-819; Lefebvre et al., 2010, PNAS 107:2343-2348). Bhat et al. reported that plant membrane proteins such as the Barley MILDEW-RESISTANCE-PROTEIN-0 (MLO), the barley syntaxin ROR2 and the Arabidopsis syntaxin PENT redistribute towards Blumeria graminis f. sp. hordei penetration points during infection (Bhat et al., 2005, PNAS 102:3135). These penetration points are strongly stained by the filipin dye, indicating abundance in sterols and leading the authors to propose that membrane-raft like domain form below mildew appressoria (Bhat et al., 2005, PNAS 102:3135; Bhat and Panstruga, 2005, Planta 223:5-19). Remarkably, species in the peronosporales are probably unable to synthesize sterols since they lack the corresponding biosynthetic enzymes (Beakes et al., 2012, Protoplasma 249:3-19). As a result, sterols forming raft-like membrane domains at the plant-pathogen interface are necessarily of plant origin. This implies that pathogens exploit plant lipid metabolism to their benefit, presumably via the action of effectors. It also implies that these pathogens are dependent on the host lipid metabolism to be able to infect. This raises the question of what could be the evolutionary advantage of pathogen's dependency on host lipid metabolism. It should be noted that the interface between host and pathogen brings into close proximity two lipid bilayers: the EHM on the host side, and the haustorium PM on the pathogen side. The composition of the EHM, distinct from any pathogen membrane, may provide a basis for the directionality of the transfer of molecules, nutrients and effectors notably, occurring through these two membranes. In addition, sterols and sphingolipids, the major lipid components of membrane rafts, are very diverse lipid groups including several plant-specific forms (Suzuki and Muranaka, 2007, Lipids 42:47-54; Pata et al., 2010, New Phytol. 185:611-630; Cacas et al., 2012, Prog. in Lipid Res. 51:272-299). These lipids may therefore constitute a signature of the host membrane that haustoria-forming pathogens evolved to recognize and manipulate specifically.

The EHM domains containing StREM1.3 co-localize with the RXLR effector AVRb1b2. Functional analysis of P. infestans AVRb1b2 effector demonstrated that host PM targeting is crucial for the promotion of susceptibility by this effector (Bozkurt 2011). Similarly, the localization of P. sojae RXLR effector Avh241 at the host PM is required for its cell-death eliciting activity (Yu et al., 2012, New Phytol. 196:247-260). Targeting of the host PM by effectors therefore appears as an important determinant of virulence. A number of effectors of bacterial pathogens are known to alter the host PM directly through lipids or via PM proteins (Ham et al., 2009, Nature Rev. Microbiol. 9:635-646). Do oomycete effectors directly target the host PM? P. infestans elicitin proteins exhibit sterol-binding properties (Kamoun et al., 1994, Appl. Environ. Microbiol. 60:1593-1598; Mikes et al., 1997, FEBS Lett. 416:190-192; Ricci, 1997, “Induction of the hypersensitive response and systemic acquired resistance by fungal proteins: the case of elicitins,” In: Stacey G, Keen NT, editors. Plant-Microbe Interactions, 3.3, Chapman & Hall, New York, pp. 53-75) that may specifically alter sterol-rich plant membrane domains. P. cryptogea cryptogein is an elicitin that triggers plant responses in a sterol-binding-dependent manner (Osman et al., 2001, Mol. Biol. Cell 12:2825-2834), including clathrin-mediated endocytosis (Leborgne-Castel et al., 2008, Plant Physiol. 146:1255-1266). Effectors such as elicitins may therefore trigger endocytosis specifically along the EHM, providing a mean for pathogens to control the homeostasis of the EHM. In addition, P. infestans RXLR effector AVR3a has lipid binding ability (Yaeno et al., 2011, PNAS 108:14682-14687; Wawra et al., 2012, J. Biol. Chem. 287: 38101-38109). Recognition of AVR3a by the cognate resistance protein R3a triggers endocytosis required for resistance (Engelhardt et al., 2012, Plant Cell 24:5142-5158). AVRb1b2 prevents secretion of the C14 defense protease, probably during release or fusion of secretory vesicles to the EHM (Bozkurt et al., 2011, PNAS 108:20832-20837). These findings point towards a critical role for the control of vesicle trafficking at the host PM for the establishment of virulence. In addition, oomycete effectors could trigger host PM reorganization into coalesced membrane rafts such as reported for some proteinaceous toxins (Garcia-Saez et al., 2011, J. Biol. Chem. 286:37768-37777). Proteins in the Remorin family were proposed to control PM lateral organization (Jarsch and Ott, 2011, Mol. Plant Microbe In. 24:7-12) and their accumulation may facilitate the action of membrane targeted effectors or drive the segregation of effectors into specific membrane domains. The finding as disclosed herein that AVRb1b2 effector co-localizes with the host susceptibility protein StREM1.3 supports the hypothesis that filamentous plant pathogen effectors exploit host membrane lateral organization to accommodate infection structures (Bhat et al., 2005, PNAS 102:3135; Caillaud et al., 2012, Plant J. 69:252-265).

Although StREM1.3 localizes in domains at the EHM from 4 dpi, it is depleted from the PM near penetration points earlier during the interaction. StREM1.3 dynamic localization in infected cells is consistent with the view that the EHM is not an extension of the host PM but rather a novel specialized membrane compartment (Koh et al., 2005, Plant J. 44:516-529; Caillaud et al., 2012, Plant J. 69:252-265; Lu et al., 2012, Cell. Microbiol. 14:682-697; Bozkurt et al., 2012, Curr. Opin. Plant Biol. 15:483-492). This also shows that although in continuity with the host PM, the EHM maintains a specific composition throughout the infection. The connection between PM and EHM occurs at the site where haustoria connect with the mycelium of the pathogen, a region called the “haustorial neckband”, believed to play a crucial role in limiting lateral diffusion between the EHM and the host PM. Koh et al. (2005, Plant J. 44:516-529) proposed two models by which the EHM could form: in a first model, vesicle fusion occurs homogeneously throughout the host cell PM and the EHM forms by invagination of the PM, with the haustorial neckband selectively filtering PM proteins from the invaginating membrane leading to differentiation of the EHM. In a second model, the EHM is built independently of the PM by targeted secretion of specialized vesicles and diffusion between EHM and PM is restricted by the haustorial neckband. Our observation that YFP:StREM1.3 is initially depleted from the PM in proximity with P. infestans penetration points suggest that Remorin does not diffuse laterally from the PM to the EHM. By contrast, AVRb1b2 effector accumulates at the EHM as early as 2 dpi, suggesting that StREM1.3 may initially be excluded from the EHM, and recruited to the EHM at later stages of the infection. This may reflect the antagonistic effect of plant and pathogen processes regulating host membrane lateral organization, raising the possibility that effectors could recruit StREM1.3 at the EHM from 4 dpi directly by binding to it or indirectly by modifying phospholipid composition of the EHM. The late recruitment of StREM1.3 at the EHM may also be associated with a change in the repertoire of effectors produced by the P. infestans, such as during the switch from biotrophy to necrotrophy. StREM1.3 accumulation at the EHM therefore likely results from selective secretion towards the haustorium, consistent with the “vesicle fusion” model of Koh et al. However, StREM1.3 is not a transmembrane protein and anchors in the PM through direct specific lipid binding (Perraki 2012) and the possibility that StREM1.3 accumulates at the EHM because of specific binding to lipids enriched in the EHM instead of selective targeting of StREM1.3-containing vesicles to the EHM remains valid. StREM1.3 redistribution towards the EHM could either directly follow StREM1.3 neo-synthesis or recycling of StREM1.3 residing at the host PM close to P. infestans penetration points. This later hypothesis is consistent with the observation that endocytosis is involved in haustoria accommodation (Hoefle et al., 2011, Plant Cell 23:2422-2439; Lu et al., 2012, Cell. Microbiol. 14:682-697).

As opposed to oomycete haustoria, haustoria of rust fungi and mildews branch and show secondary extensions similar to symbiotic arbuscules and the overall morphology of the EHM differs between species (e.g. Mims et al., 2004, Can. J. Botany 82:1001-1008; Avrova et al., 2008, Cell. Microbiol. 10:2271-2284; Spanu et al., 2010, Science 330:1543). The protein composition of the EHM also varies depending on the host and pathogen species: the A. thaliana flagellin receptor FLS2 was found in the EHM of A. thaliana cells infected by H. arabidopsidis but not in the EHM of N. benthamiana cells infected by P. infestans (Lu et al., 2012, Cell. Microbiol. 14:682-697). The mechanisms leading to formation of the EHM therefore seems to vary according to the plant and pathogen partners involved, possibly in relation with pathogens lifestyle, implying different requirements for suppression of host immunity (Lu et al., 2012, Cell. Microbiol. 14:682-697; Kemen and Jones, 2012, Trends Plant Sci. 17:448-457). Such diversity in the nature of EHMs may involve specific mechanisms for differentiation and the question of the specificity of StREM1.3 association with the EHM and with susceptibility to filamentous plant pathogens remains open.

### Materials and Methods

### Plant Lines and Growth Conditions

Leaves from five week old N. benthamiana and tomato (Solanum lycopersicum cv Ailsa Craig) grown in a growth chamber at 25° C. under 16/8 h day/night conditions were used for all experiments. 35S-YFP:StREM1.3 transgenic N. benthamiana plants were obtained from (Lu et al., 2012, Cell. Microbiol. 14:682-697), T2 plants were screened using YFP fluorescence observed under a confocal microscope. Sense and antisense SlREM1.2 tomato plants were obtained from (Raffaele et al., 2009, Plant Cell 21:1541-1555). All tomato plants used were T3 and T4 plants and were screened by protein gel blot analysis using anti-Remorin (Raffaele et al., 2009, Plant Cell 21:1541-1555) antibodies. Protein Blot signal was quantified using the gel analysis function in ImageJ program and only plants showing Remorin level<80% and >150% of wild type level were considered as antisense and sense plants respectively.

### Cloning Procedures and Plasmid Constructs

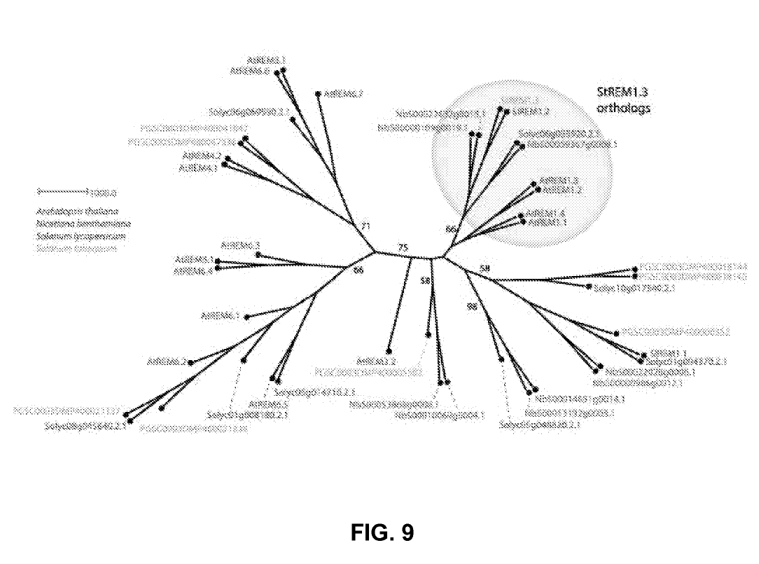

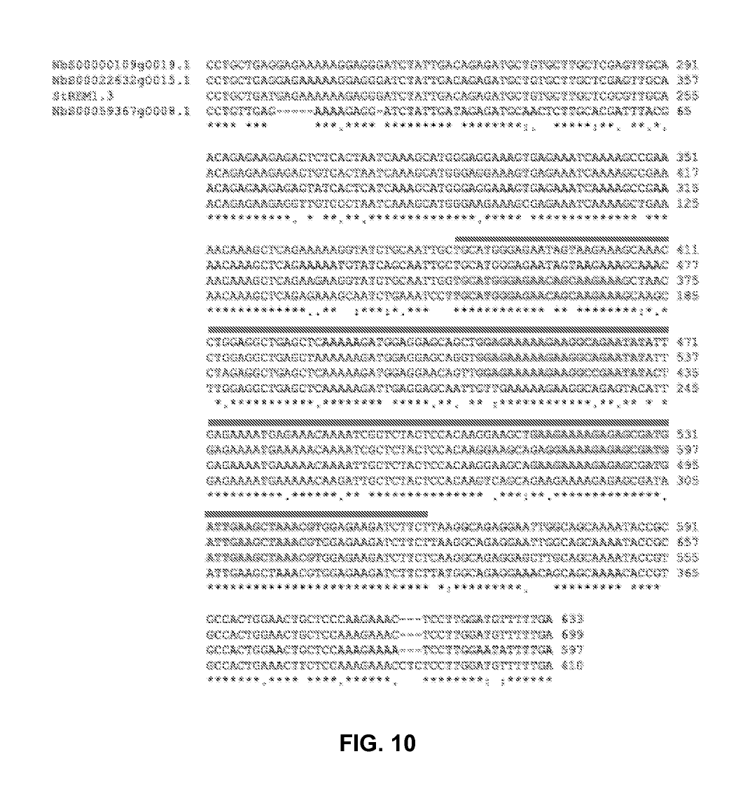

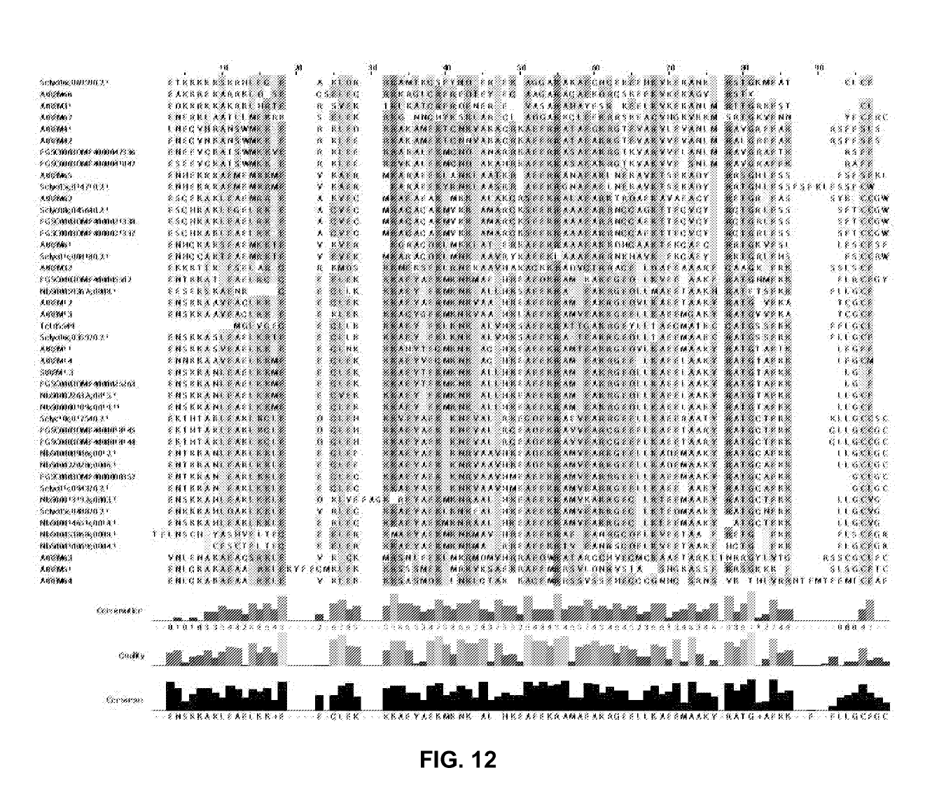

The 35S-YFP:StREM1.3 construct was obtained from (Raffaele et al., 2009, Plant Cell 21:1541-1555), The 35S-RFP:Avrblb2 construct from (Bozkurt et al., 2011, PNAS 108:20832-20837), the 35S-YFP:StREM1.3* and 355-YFP:StREM1.3ΔCA constructs from (Perraki et al., 2012, Plant Physiol. 160:624-637) and the GFP:HaRXL17 from (Caillaud et al., 2012, Plant J. 69:252-265). The 35S-RFP:StREM1.3 was generated using classical Gateway cloning into the pH7WGR2 vector (Karimi et al., 2002, Trends Plant Sci. 7:193-195). The 35S-GFP:SYT1 and 35S-RFP:SYT1 constructs were generated from specific amplification of N. benthamiana cDNAs with the 5′-AAAAAGCAGGCTTCATGGGTTTTGTGAGTACTATA-3′ (SEQ ID NO: 9) and 5′ AGAAAGCTGGGTCTCATGATGCAGTTCTCCATTG-3′ (SEQ ID NO: 10) primers and classical Gateway cloning into the pk7WGF2 and pH7WGR2 vectors. To design the remorin silencing construct, we first performed a phylogenetic analysis on Remorin_C domains using remorin sequences identified in N. benthamiana (9), tomato (11), potato (9) and Arabidopsis thaliana (16) whole genomes (FIG. 9). A 101 amino-acids alignment of a conserved region was constructed using MUSCLE and used as input in Phylip (Felseinstein 1989) to build a consensus parsimony tree after 100 replicates bootstrap analysis (FIGS. 9, 12). This analysis revealed three members of the StREM1.3 clade in N. benthamiana. We identified a silencing construct covering 178 nucleotides at the C-terminus of StREM1.3 allowing to specifically silence StREM1.3 and its three homologs in N. benthamiana (FIG. 10). The Remorin VIGS silencing construct was generated by PCR amplification using full length StREM1.3 as a template with forward primers including a BamHI restriction site and reverse primers including a KpnI restriction site. PCR products were digested with BamHI and KpnI and ligated into the A. tumefaciens binary tobravirus vector pTV00 (Ratcliff et al., 2001, Plant J. 25:237-245). Silencing experiments were performed as described in (Bos et al., 2010, PNAS 107:9909-9914) using pTV00 empty vector as a negative control and pTV00 carrying N. benthamiana phytoene desaturase gene fragment as a silencing control. Remorin silencing was verified by loss of fluorescence in 35S-YFP:StREM1.3 stable transgenic plants and anti-Remorin Western Blot.

### Transient Expression in Planta

A. tumefaciens GV3101 was used to deliver T-DNA constructs into 3-week-old N. benthamiana plants. Overnight A. tumefaciens cultures were harvested by centrifugation at 10,000 g, resuspended in infiltration medium [10 mM MgCl2, 5 mM 2-(N-morpholine)-ethanesulfonic acid (MES), pH 5.3, and 150 mM acetosyringone] prior to syringe infiltration into either the entire leaf or leaf sections. For confocal microscopy, constructs were infiltrated to a final OD₆₀₀=0.4, in equal amounts in the case of co-infiltrations. For transient protein expression followed by P. infestans inoculation, the constructs were infiltrated to an OD600=0.3 supplemented with p19 silencing suppressor to an OD₆₀₀=0.1, and P. infestans was inoculated 24 hours later. For VIGS silencing pTV00 and pBINTRA constructs were co-infiltrated at OD₆₀₀=0.3 and OD₆₀₀=0.2 respectively.

### Confocal Microscopy

Imaging was performed on a Leica TCS SP5 confocal microscope (Leica Microsystems, Germany) using 20×, 40× air and 63× water immersion objectives. Excitation wavelengths and filters for emission spectra were set as described in (Lu 2012). Co-localization images were taken using sequential scanning between lines. Image analysis was done with the Leica LAS AF software, ImageJ (1.43u) and Adobe PHOTOSHOP CS4 (11.0). Callose staining and imaging was performed as described in (Bozkurt 2011).

### Pathogenicity Assays

Unless stated otherwise, P. infestans infection assays were performed by inoculation with 10 μl droplets of zoospore solutions at 50.μL-1 zoospores on detached N. benthamiana leaves (Chaparro-Garcia 2011). P. infestans isolate 88069 (van West et al., 1999, Mol. Cell 3:339-348) and a transformant expressing a cytosolic tandem DsRed protein (88069td) (Chaparro-Garcia et al., 2011, PLoS One 6:e16608). For transient protein expression followed by P. infestans inoculation, constructs were expressed by Agrobacterium-mediated transformation together with p19 silencing suppressor 24 hours prior to P. infestans inoculation. Lesion sizes were calculated on pictures analyzed using area measurements in ImageJ (1.43u).

### Protein Extraction and Immunoblots

Proteins were transiently expressed by A. tumefaciens in N. benthamiana leaves and harvested two days post infiltration. Protein extracts were prepared by grinding leaf samples in liquid nitrogen and extracting 1 gram of tissue in 3 ml GTEN protein extraction buffer (150 mM Tris-HCl pH 7.5; 150 mM NaCl; 10% glycerol; 10 mM EDTA) and freshly added 10 mM DTT; 2% (w/v) PVPP; 1% (v/v) protease inhibitor cocktail (Sigma); 1% (v/v) NP-40 according to (win 2011). Anti-Remorin (Raffaele et al., 2009, Plant Cell 21:1541-1555) and commercial anti-GFP (Invitrogen) were used as primary antibodies. Western Blot signal was quantified using Gel analysis in ImageJ (1.43u) and normalized based on the quantification of total proteins stained by Ponceau red.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for enhancing the resistance of a plant to an oomycete plant pathogen, the method comprising decreasing the level and/or activity of a remorin in the plant or part thereof, wherein the remorin is a remorin that is found in an extrahaustorial membrane (EHM) that is formed in the plant in response to the oomycete plant pathogen.

2. The method of claim 1, wherein the remorin is selected from the group consisting of StREM1.3 and SlREM1.2.

3. The method of claim 1 or 2, wherein decreasing the level and/or activity of the a remorin in the plant or part thereof comprises introducing a polynucleotide construct into at least one plant cell, the polynucleotide comprising a promoter operably linked to a transcribed region, wherein the promoter is expressible in a plant cell, and wherein the transcribed region is designed to produce a transcript for antisense-mediated gene silencing or post-transcriptional gene silencing of the remorin.

4. The method of claim 3, wherein the polynucleotide construct is stably incorporated into the genome of the plant cell.

5. The method of claim 3 or 4, wherein the plant cell is regenerated into a plant comprising in its genome the polynucleotide construct.

6. The method of claim 3, the polynucleotide construct is not stably incorporated into the genome of the plant.

7. The method of claim 6, wherein the polynucleotide construct is in a viral vector.

8. The method of claim 7, wherein the viral vector is a tobacco rattle virus vector.

9. The method of claim 8, wherein is a tobacco rattle virus vector is pTV00.

10. The method of any one of claims 3-9, wherein the promoter is selected from the group consisting of pathogen-inducible, constitutive, tissue-preferred, wound-inducible, and chemical-regulated promoters.

11. The method of any one of claims 1-10, wherein the remorin is StREM1.3.

12. The method of claim 11, wherein the transcribed region comprises the nucleotide sequence set forth in SEQ ID NO: 8.

13. The method of claim 1 or 2, wherein decreasing the level and/or activity of the a remorin in the plant or part thereof comprises disrupting in a plant cell a remorin gene, wherein the disruption decreases the level and/or activity of the remorin in the plant cell compared to a corresponding control plant cell lacking disruption of the remorin gene.

14. The method of claim 13, wherein disrupting comprises an insertion, a deletion, or a substitution of a least one base pair in the remorin gene.

15. The method of claim 14, wherein disrupting further comprises targeted mutagenesis, homologous recombination, or mutation breeding.

16. The method of any one of claims 1-15, wherein the part thereof is an EHM.

17. The method of any one of claims 1-15, wherein the part thereof is selected from the group consisting of a leaf, a stem, a tuber, and a fruit.

18. The method of any of one of claims 1-15, wherein the part thereof is a plant cell.

19. The method of any one of claims 1-18, wherein the plant is a Solanaceous plant.

20. The method of claim 19, wherein the Solanaceous plant is selected from the group consisting of potato, tomato, eggplant, pepper, tobacco, and petunia.

21. The method of any one of claims 1-18, wherein the plant is selected from the group consisting of potato, eggplant, pepper, tobacco, petunia, lettuce, pea, bean, spinach, melon, cucumber, squash, Brassica sp., radish, onion, and watermelon.

22. The method of any one of claims 1-21, wherein the level and/or activity of the remorin in the plant or the part thereof is decreased when compared to the level and/or activity of the remorin in a control plant or the corresponding part of the control plant.

23. The method of any one of claims 1-22, wherein the plant comprises enhanced resistance to the oomycete plant pathogen when compared to the resistance of a control plant to the oomycete plant pathogen.

24. The method of any one of claims 1-23, further comprising decreasing the level and/or activity of at least one additional remorin in the plant or part thereof, wherein the level and/or activity of the at least one additional remorin is decreased when compared to the level and/or activity of the at least one additional remorin in a control plant.

25. The method of any one of claims 1-24, wherein the oomycete pathogen is selected from the group consisting of Phytophthora infestans, Phytophthora ipomoeae, Phytophthora mirabilis, Phytophthora phaseoli, Phytophthora capsici, Phytophthora porri, Phytophthora parasitica, Phytophthora ipomoeae, Phytophthora mirabilis, Hyaloperonospora arabidopsidis, Peronospora farinosa, Pseudoperonospora cubensis, Hyaloperonospora parasitica, Peronospora destructor, Bremia lactucae, Pseudoperonospora cubensis, Pseudoperonospora humuli, Peronospora destructor, Albugo candida, Albugo occidentalis, and Pythium spp.

26. A plant with enhanced resistance to an oomycete plant pathogen, the plant comprising a mutation in a remorin gene, wherein the plant has a decreased level and/or activity of remorin in the plant or part thereof as compared to a control plant that lacks enhanced resistance to the oomycte plant pathogen.

27. The plant of claim 26, wherein the mutation is a non-naturally occurring mutation.

28. The plant of claim 26 or 27, wherein the mutation comprises an insertion, a deletion, or a substitution of a least one base pair in the remorin gene.

29. The plant of any one of claims 26-28, wherein the plant is non-transgenic or transgenic.

30. The plant of any one of claims 26-29, wherein the plant is selected from the group consisting of potato, eggplant, pepper, tobacco, petunia, lettuce, pea, bean, spinach, melon, cucumber, squash, Brassica sp., radish, onion, and watermelon.

31. The plant of claim 30, wherein the plant is potato and the remorin is StREM1.3.

32. The plant of claim 30, wherein the plant is tomato and the remorin is SlREM1.2.

33. A method of producing a plant with enhanced resistance to an oomycete plant pathogen, the method comprising stably incorporating in the genome of at least one plant cell a polynucleotide construct comprising a promoter operably linked to a transcribed region, wherein the promoter is expressible in a plant cell, and wherein the transcribed region is designed to produce a transcript for antisense-mediated gene silencing or post-transcriptional gene silencing of a remorin that is found in an extrahaustorial membrane (EHM) that is formed in the plant in response to the oomycete plant pathogen.

34. The method of claim 33, wherein the remorin is selected from the group consisting of StREM1.3 and SlREM1.2.

35. The method of claim 33 or 34, wherein the plant cell is regenerated into a plant comprising in its genome the polynucleotide construct.

36. The method of any one of claims 33-35, wherein the level and/or activity of the remorin in the plant or part thereof is decreased when compared to the level and/or activity of the remorin in a control plant or the corresponding part of the control plant.

37. The method of any one of claims 33-36, wherein the plant comprises enhanced resistance to the oomycete plant pathogen when compared to the resistance of a control plant to the oomycete plant pathogen.

38. The method of claim 36 or 37, wherein the part thereof is an EHM.

39. The method of claim 36 or 37, wherein the part thereof is selected from the group consisting of a leaf, a stem, a tuber, and a fruit.

40. The method of claim 36 or 37, wherein the part thereof is a plant cell.

41. The method of any one of claims 33-40, wherein the remorin is StREM1.3.

42. The method of claim 41, wherein the transcribed region comprises the nucleotide sequence set forth in SEQ ID NO: 8.

43. The method of any one of claims 33-42, wherein the promoter is selected from the group consisting of pathogen-inducible, constitutive, tissue-preferred, wound-inducible, and chemical-regulated promoters.

44. The method of any one of claims 33-43, wherein the plant is a Solanaceous plant.

45. The method of claim 44, wherein the Solanaceous plant is selected from the group consisting of potato, tomato, eggplant, pepper, tobacco, and petunia.

46. The method of any of claims 33-44, wherein the plant is selected from the group consisting of potato, eggplant, pepper, tobacco, petunia, lettuce, pea, bean, spinach, melon, cucumber, squash, Brassica sp., radish, onion, and watermelon.

47. The method of any one of claims 33-46, further comprising stably incorporating in the genome of the at least one plant cell an additional polynucleotide construct comprising a promoter operably linked to a transcribed region, wherein the second transcribed region is designed to produce a transcript for antisense-mediated gene silencing or post-transcriptional gene silencing of a second remorin that is found in an extrahaustorial membrane (EHM) that is formed in the plant in response to the oomycete plant pathogen.

48. The method of any one of claims 33-47, wherein the oomycete pathogen is selected from the group consisting of Phytophthora infestans, Phytophthora ipomoeae, Phytophthora mirabilis, Phytophthora phaseoli, Phytophthora capsici, Phytophthora porri, Phytophthora parasitica, Phytophthora ipomoeae, Phytophthora mirabilis, Hyaloperonospora arabidopsidis, Peronospora farinosa, Pseudoperonospora cubensis, Hyaloperonospora parasitica, Peronospora destructor, Bremia lactucae, Pseudoperonospora cubensis, Pseudoperonospora humuli, Peronospora destructor, Albugo candida, Albugo occidentalis, and Pythium spp.

49. A transformed plant comprising stably incorporated in its genome a polynucleotide construct comprising a promoter operably linked to a transcribed region, wherein the promoter is expressible in a plant cell, and wherein the transcribed region is designed to produce a transcript for antisense-mediated gene silencing or post-transcriptional gene silencing of a remorin that is found in an extrahaustorial membrane (EHM) that is formed in the plant in response to the oomycete plant pathogen.

50. The transformed plant of claim 49, wherein the remorin is selected from the group consisting of StREM1.3 and SlREM1.2.

51. The transformed plant of claim 49 or 50, wherein the level and/or activity of the remorin in the plant or part thereof is decreased when compared to the level and/or activity of the remorin in a control plant or the corresponding part of the control plant.

52. The transformed plant of any one of claims 49-51, wherein the plant comprises enhanced resistance to the oomycete plant pathogen when compared to the resistance of a control plant to the oomycete plant pathogen.

53. The transformed plant of claim 51 or 52, wherein the part thereof is an EHM.

54. The transformed plant of any one of claim 51 or 52, wherein the part thereof is selected from the group consisting of a leaf, a stem, a tuber, and a fruit.

55. The transformed plant of any one of claim 51 or 52, wherein the part thereof is a plant cell.

56. The transformed plant of any one of claims 49-55, wherein the remorin is StREM1.3.

57. The transformed plant of any one of claims 49-56, wherein the transcribed region comprises the nucleotide sequence set forth in SEQ ID NO: 8.

58. The transformed plant of any one of claims 49-57 wherein the promoter is selected from the group consisting of pathogen-inducible, constitutive, tissue-preferred, wound-inducible, and chemical-regulated promoters.

59. The transformed plant of any one of claims 49-58, wherein the plant is a Solanaceous plant.

60. The transformed plant of claim 59, wherein the Solanaceous plant is selected from the group consisting of potato, tomato, eggplant, pepper, tobacco, and petunia.

61. The transformed plant of any of claims 49-59, wherein the plant is selected from the group consisting of potato, eggplant, pepper, tobacco, petunia, lettuce, pea, bean, spinach, melon, cucumber, squash, Brassica sp., radish, onion, and watermelon.

62. The transformed plant of any of claims 49-61, wherein the transformed plant is a seed or a tuber comprising the polynucleotide construct.

63. The transformed plant of any one of claims 49-62, wherein the oomycete pathogen is selected from the group consisting of Phytophthora infestans, Phytophthora ipomoeae, Phytophthora mirabilis, Phytophthora phaseoli, Phytophthora capsici, Phytophthora porri, Phytophthora parasitica, Phytophthora ipomoeae, Phytophthora mirabilis, Hyaloperonospora arabidopsidis, Peronospora farinosa, Pseudoperonospora cubensis, Hyaloperonospora parasitica, Peronospora destructor, Bremia lactucae, Pseudoperonospora cubensis, Pseudoperonospora humuli, Peronospora destructor, Albugo candida, Albugo occidentalis, and Pythium spp.

64. A fruit, seed, or tuber produced the plant of any one of claims 26-32 and 49-63.

65. A food product produced using the fruit, seed, or tuber of claim 64.

66. A method of limiting disease caused by an oomycete pathogen in agricultural crop production, the method comprising planting the plant according to any one of claims 26-32 and 49-63 and exposing the plant to conditions favorable for growth and development of the transformed plant.

67. The method of claim 66, wherein the plant is grown outdoors or in a greenhouse.

68. The method of claim 66 or 67, further comprising harvesting an agricultural product produced by the transformed plant.

69. The method of claim 68, wherein the product is a fruit, a leaf, or a tuber.

70. Use of the plant of any one of claims 26-32 and 49-63 in agriculture.

71. The use of claim 70, wherein the plant is a seed or a tuber.


SEQUENCE LISTING (10 sequences)

SEQ ID NO: 1 - DNA, 597 bp, Solanum tuberosum
  CDS (1)..(597)
atggcagaattggaagctaagaaagtagaaattgtggaccctgcaccccctgcgccaggacctgttgaagctcctaaagaagtggtggctgatgagaaagccatagttgcaccagctctgcctcctcctgcagaagaaaaagaaaaacccgatgactcgaaagcattagttgtcgttgaaactaaagcaccagaacctgctgatgagaaaaaagagggatctattgacagagatgctgtgcttgctcgcgttgcaacagagaagagagtatcactcatcaaagcatgggaggaaagtgagaaatcaaaagccgaaaacaaagctcagaagaaggtatctgcaattggtgcatgggagaacagcaagaaagctaacctagaggctgagctcaaaaagatggaggaacagttggagaaaaagaaggccgaatatactgagaaaatgaaaaacaaaattgctctactccacaaggaagcagaagaaaagagagcgatgattgaagctaaacgtggagaagatcttctcaaggcagaggagcttgcagcaaaataccgtgccactggaactgctccaaagaaaatccttggaatattttga

SEQ ID NO: 2 - protein, 198 aa, Solanum tuberosum
MAELEAKKVEIVDPAPPAPGPVEAPKEVVADEKAIVAPALPPPAEEKEKPDDSKALVVVETKAPEPADEKKEGSIDRDAVLARVATEKRVSLIKAWEESEKSKAENKAQKKVSAIGAWENSKKANLEAELKKMEEQLEKKKAEYTEKMKNKIALLHKEAEEKRAMIEAKRGEDLLKAEELAAKYRATGTAPKKILGIF

SEQ ID NO: 3 - DNA, 594 bp, Lycopersicon esculentum
  CDS (1)..(594)
atggcagaattggaagctaagaaagtagaaattgtggaccctgcccctgcacaagaaccagttgaagctcctaaagaagtggtggctgatgagaaagccatagttgaaccagctccgcctcctcctgcagaagaaaaagaaaaacccgatgactcgaaagcactagttgttgtcgaaaataaagcagaagaagctgctgatgagaaaaaagagggatctattgatagagatgctgtgcttgcacgcgttgcaactgagaagaggctatcactcatcaaagcatgggaagaaagtgagaaatcaaaagccgaaaacaaagctcagaagaaggtgtctgcaattggtgcatgggagaacagcaagaaagcaaacctagagtctgagctcaaaaagatggaggaacagttggagaaaaagaaggcaatatatactgagaaaatgaaaaacaaaattgctctactccacaaggaagcagaagaaaagagagcgatgattgaagctaaacgtggagaagatcttctcaaggcagaggagcttgcagcaaaataccgcgccactggaactgctccaaagaaaatccttggaatattttga

SEQ ID NO: 4 - protein, 197 aa, Lycopersicon esculentum
MAELEAKKVEIVDPAPAQEPVEAPKEVVADEKAIVEPAPPPPAEEKEKPDDSKALVVVENKAEEAADEKKEGSIDRDAVLARVATEKRLSLIKAWEESEKSKAENKAQKKVSAIGAWENSKKANLESELKKMEEQLEKKKAIYTEKMKNKIALLHKEAEEKRAMIEAKRGEDLLKAEELAAKYRATGTAPKKILGIF

SEQ ID NO: 5 - DNA, 620 bp, Artificial Sequence
  Sense SlREM1.2 construct
gtttgtagccatggcagaattggaagctaagaaagtagaaattgtggaccctgcccctgcacaagaaccagttgaagctcctaaagaagtggtggctgatgagaaagccatagttgaaccagctccgcctcctcctgcagaagaaaaagaaaaacccgatgactcgaaagcactagttgttgtcgaaaataaagcagaagaagctgctgatgagaaaaaagagggatctattgatagagatgctgtgcttgcacgcgttgcaactgagaagaggctatcactcatcaaagcatgggaagaaagtgagaaatcaaaagccgaaaacaaagctcagaagaaggtgtctgcaattggtgcatgggagaacagcaagaaagcaaacctagagtctgagctcaaaaagatggaggaacagttggagaaaaagaaggcaatatatactgagaaaatgaaaaacaaaattgctctactccacaaggaagcagaagaaaagagagcgatgattgaagctaaacgtggagaagatcttctcaaggcagaggagcttgcagcaaaataccgcgccactggaactgctccaaagaaaatccttggaatattttgaagcagcaagtaccagg

SEQ ID NO: 6 - DNA, 597 bp, Artificial Sequence
  Antisense SlREM1.2 construct
ttcaaaatattccaaggattttctttggagcagttccagtggcgcggtattttgctgcaagctcctctgccttgagaagatcttctccacgtttagcttcaatcatcgctctcttttcttctgcttccttgtggagtagagcaattttgtttttcattttctcagtatatattgccttctttttctccaactgttcctccatctttttgagctcagactctaggtttgctttcttgctgttctcccatgcaccaattgcagacaccttcttctgagctttgttttcggcttttgatttctcactttcttcccatgctttgatgagtgatagcctcttctcagttgcaacgcgtgcaagcacagcatctctatcaatagatccctcttttttctcatcagcagcttcttctgctttattttcgacaacaactagtgctttcgagtcatcgggtttttctttttcttctgcaggaggaggcggagctggttcaactatggctttctcatcagccaccacttctttaggagcttcaactggttcttgtgcaggggcagggtccacaatttctactttcttagcttccaattctgccatgg

SEQ ID NO: 7 - DNA, 740 bp, Artificial Sequence
  Duplicated 35S CaMV (Cabb B-JI isolate); 35SS
cctactccaaaaatgtcaaagatacagtctcantaagnaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagccc

SEQ ID NO: 8 - DNA, 177 bp, Artificial Sequence
  Remorin StREM1.3 virus-induced gene silencing (VIGS) construct
tgcatgggagaacagcaagaaagcaagcttggaggctgagctcaaaaagattgaggagcaattgttgaaaaagaaggcagagtacattgagaaaatgaaaaacaagattgctctactccacaagtcagcagaagaaaagagagcgataattgaagctaaacgtggagaagatcttct

SEQ ID NO: 9 - DNA, 35 bp, Artificial Sequence
  PCR Primer
aaaaagcaggcttcatgggttttgtgagtactata

SEQ ID NO: 10 - DNA, 34 bp, Artificial Sequence
  PCR Primer
agaaagctgggtctcatgatgcagttctccattg